(19) United States
(12) Patent Application Publication
(10) Pub. No.: US 2024/0368065 A1
(43) Pub. Date: Nov. 7, 2024

(54) CITRACONIC ACID AND DERIVATIVES THEREOF FOR USE AS A MEDICAMENT

(71) Applicant: HELMHOLTZ-ZENTRUM FÜR INFEKTIONSFORSCHUNG GMBH, Braunschweig (DE)

(72) Inventors: Frank PESSLER, Braunschweig (DE); Fangfang CHEN, Braunschweig (DE); Moritz WINTERHOFF, Braunschweig (DE); Konrad BÜSSOW, Braunschweig (DE); Wulf BLANKENFELDT, Braunschweig (DE); Anna HIRSCH, Saarbrücken (DE); Walid A. M. ELGAHER, Saarbrücken (DE)

(21) Appl. No.: 18/556,649

(22) PCT Filed: Apr. 22, 2022
(86) PCT No.: PCT/EP2022/060682

§ 371 Date: Oct. 20, 2023

(30) Foreign Application Priority Data
Apr. 23, 2021 (EP) 21170041.4
Oct. 15, 2021 (EP) 21202816.1

(51) Int. Cl. C07C 57/42; A61K 31/122; A61K 31/194; A61P 31/16; C07C 61/20; C07C 69/593

(52) U.S. Cl. CPC C07C 57/42; A61K 31/122; A61K 31/194; A61P 31/16; C07C 61/20; C07C 69/593

(57) ABSTRACT

Citraconic acid (CA) or derivative thereof for use as medicament, for example in the treatment of a medical condition associated with inflammation. In another aspect, the invention relates to a citraconic acid derivative according to formulae 1-XIII. In another aspect, the invention relates to a pharmaceutical composition comprising citraconic acid or derivative thereof with one or more pharmaceutical excipients for use in the treatment of a medical condition, such as those associated with inflammation and/or a non-inflammation associated medical condition.

The present invention is in the field of pharmaceutical substances and compositions, in particular immunomodulators, and their corresponding medical use.

In one aspect, the invention relates to citraconic acid (CA) or derivative thereof for use as a medicament. In some embodiments, the citraconic acid or derivative thereof is intended for use in the treatment of a medical condition associated with inflammation. In some embodiments, the citraconic acid derivative for use is according to formula I. In some embodiments, the citraconic acid derivative for use is according to formula VII.

In one aspect, the invention relates to citraconic acid or derivative thereof according to formula VII and said compounds for use as a medicament as described herein

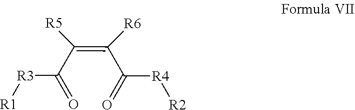

wherein R1 and R2 are, independently, H, a branched or straight chain alkyl, alkenyl, alkynyl, aryl, cycloalkyl or alkoxy group of C1-C12, preferably C1-C8, more preferably C1-C8 alkyl, wherein R1 and/or R2 is optionally substituted with halogen, alkoxy, hydroxy, amine or C1-C8 alkyl,
wherein R3 and R4 are, independently, a heteroatom, preferably O, S or N.
wherein R5 and R6 are independently, H, a branched or straight chain alkyl, alkenyl, alkynyl, aryl, heteroaryl, cycloalkyl or alkoxy group of C1-C12, preferably C1-C8, more preferably C1-C4 alkyl or phenyl, wherein R5 and/or R6 is optionally substituted with halogen, alkoxy, hydroxy, amine or C1-C8 alkyl,
wherein optionally, R5 is fused to R6 to form a cyclic structure, preferably a 5-or 6-membered structure, preferably a cyclopentene.

In one aspect, the invention relates to citraconic acid derivatives according to formula I and said compounds for use as a medicament as described herein

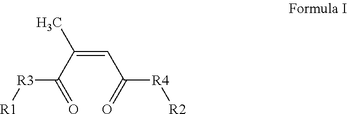

wherein R1 and R2 are, independently, H, a branched or straight chain alkyl, alkenyl, alkynyl, aryl, cycloalkyl or alkoxy group of C1-C12, preferably C1-C8, more preferably C1-C8 alkyl, wherein R1 and/or R2 is optionally substituted with halogen, alkoxy, hydroxy, amine or C1-C8 alkyl,
and wherein R3 and R4 are, independently, a heteroatom, preferably O, S or N.

In another aspect, the invention relates to a pharmaceutical composition comprising citraconic acid or derivative thereof with one or more pharmaceutical excipients for use in the treatment of a medical condition, such as those associated with inflammation.

### BACKGROUND OF THE INVENTION

Inflammatory diseases include multiple disorders and conditions that are characterized by inflammation. Examples include allergy, asthma, autoimmune diseases, coeliac disease, glomerulonephritis, hepatitis, inflammatory bowel disease, reperfusion injury and transplant rejection, amongst others. Such diseases are therefore widespread. Some estimates consider up to 5% of human populations to suffer from a medical condition associated with inflammation, whereby up to 50% of all deaths are considered to be attributable to inflammation-related disease (Furman et al, Nature Medicine, 25:1822-1832, 2019). Arthritis and joint diseases alone are estimated to be present in approx. 350 million people worldwide, whereby inflammatory bowel disease (IBD) had an estimated prevalence in 2015 in the USA of 1.3% of all adults.

Immunomodulators are pharmaceutically active agents that are used to treat such diseases, including chronic diseases such as autoimmune diseases, multiple sclerosis, Crohn's disease and allergic diseases, infections that do not respond or are resistant to antibiotics, to treat cancer, asthma, as well as to optimize the effect of others drugs.

Immunomodulators also play an important role in the context of the COVID-19 pandemic. SARS-CoV-2 infection and lung cell destruction triggers a local immune response that recruits macrophages and monocytes that respond to infection, release cytokines, and trigger adaptive T-and B-cell immune responses. In most cases, this process can resolve the infection. However, in some cases, a dysfunctional immune response occurs that can cause severe pulmonary and even systemic pathologies. Therefore, controlling the inflammatory response may be as important as fighting the virus itself. Therapies that inhibit viral infection and regulate dysfunctional immune responses may act synergistically to block pathologies at multiple levels. The same principle applies to many other infections, by bacteria, viruses or fungi.

The use of itaconic acid as an immunomodulator is known in the art. For example, WO2017142855 discloses a method of suppressing an immune response comprising administration of an immunomodulatory agent such as itaconate, malonate, or a derivative thereof. Further, the disclosure provides a method for reducing the extent of tissue injury in ischemia reperfusion, including cardiovascular infarction comprising administration of an immunomodulatory agent and treating psoriasis.

WO2020006557 describes the use of derivatives of itaconic acid as medical agents for activating the immune system and suppressing activation of the immune system, for treating viral infections, bacterial infections, fungal infections, ischemia, sepsis, inflammatory diseases of the digestive tract or skin, bone disease, and cancer.

Swain et al (Nature Metabolism, 2:594-602, 2020) teach that itaconate treatment suppressed IL-1β secretion and strongly enhanced lipopolysaccharide-induced interferon-β secretion. Macrophages with deficient expression of IRG1, which is a key factor for itaconate production, produced lower levels of interferon and reduced transcriptional activation of this pathway. This establishes itaconate as an immunoregulatory, rather than strictly immunosuppressive, metabolite.

U.S. Pat. No. 20,090,35385 discloses a composition comprising citraconic acid for use in the treatment of disorders related to iron deficiency, such as viral infections, cancer, rheumatoid arthritis. W003068209 discloses a composition comprising metformin and tocopherol acid citraconate for use in the treatment of diabetes. W02020229297 discloses citraconate derivatives for use in the treatment of inflammation, in particular rheumatoid arthritis. Citraconic acid (CA) is however only mentioned as an excipient, solubility or adsorption enhancer or in salt generation. None of the aforementioned documents make any mention of CA or derivatives thereof having anti-inflammatory properties, or of CA or derivatives thereof being administered for the purpose of directly reducing inflammation in medical applications.

Further documents describe potential CA derivatives for medical use (Pavic Kristina et al, Bioorganic & Medicinal Chemistry Letters, vol. 29, no. 19, 2019; CN106420711; WO2020/227085). However, such derivatives are structurally distinct from the CA derivatives of the present invention.

Further documents disclose potential CA derivatives, although for uses and purposes unrelated to the present invention (WO 2018/114073; Brenna Elisabetta et al, Advanced synthesis and catalysis, vol. 354, no. 14-15, 2012, Welling et al, Pesticide biochemistry and physiology, vol. 23, no. 3, 1985, Tamiri et al, International journal of mass spectrometry, vol. 228, no. 2-3, 2003; Gogoi et al, Tetrahedron, vol. 60, no. 41, 2004).

Despite the known use of immunomodulatory drugs, there remains an urgent need for alternative or improved substances to regulate various aspects of inflammation, in particular to reduce mortality in patients with tumor diseases, bacterial infection, viral infection, or other conditions associated with inflammation.

### SUMMARY OF THE INVENTION

In light of the prior art the technical problem underlying the present invention is to provide alternative and or improved substances for treating medical conditions associated with inflammatory disease.

This problem is solved by the features of the independent claims. Preferred embodiments of the present invention are provided by dependent claims.

The invention therefore relates to citraconic acid (CA) or derivatives thereof for use as medicament.

In one embodiment, citraconic acid or a derivative thereof is used in the treatment of a medical condition associated with inflammation. In other embodiments, citraconic acid or derivatives thereof is used for the treatment of a medical condition not associated with inflammation. The invention further relates to citraconic acid derivatives according to formula I or formula VII, as described below. The invention further relates to a pharmaceutical composition comprising citraconic acid or a derivative thereof with one or more pharmaceutical excipients for use in the treatment of a medical condition, preferably those associated with inflammation.

These various embodiments and aspects of the invention are unified by, benefit from, are based on and/or are linked by the common and surprising and beneficial finding that citraconic acid or derivatives thereof are suitable as a medicament, in particular for the treatment of inflammation.

In one aspect, the invention relates to citraconic acid or derivatives thereof for use as a medicament.

Citraconic acid (synonym: 2-methylmaleic acid) is the cis stereoisomer of mesaconic acid and, like the similar itaconic acid, belongs to the isomeric dicarboxylic acids derived from citric acid.

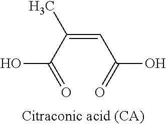

Until the present invention, to the knowledge of the inventors citraconic acid has been primarily used as an antioxidant for oils and fats. In addition, like itaconic and mesaconic acid, it has been applied as a co-monomer in the synthesis of copolymers with antiviral and antibacterial activity. To the knowledge of the inventors, citraconic acid as such has not been previously proposed for medical use.

In some embodiments, citraconic acid or derivatives thereof are considered to be present, applied and/or administered in their free form, in other words free from any given polymeric structure encompassing citraconic acid. The provision of solid forms of citraconic acid, such as pharmaceutical salts of crystalline forms, are considered to fall under the non-polymeric free form of the substance.

Citraconic acid typically forms an inner anhydride, analogously to maleic acid. Citraconic anhydride is interesting particularly because of its low melting point (easier handling at lower temperature). Many attempts have been made to develop industrial processes for obtaining citraconic anhydride, including its preparation from citric acid, and gas-phase oxidation of alkylsubstituted olefins, methylbutanols, or mesityl oxide. In the gas-phase oxidation of o-xylene, ca. 2-4 t of citraconic anhydride is formed per 1000 t of phthalic anhydride. Citraconic anhydride is present as citraconic acid after water washing. Three processes have been developed to isolate citraconic anhydride. Its synthesis from succinic anhydride and formaldehyde has also been proposed.

With regard to its industrial use, citraconic anhydride is frequently mentioned with maleic acid in the patent literature. Substitution of citraconic acid for maleic acid, however, does not impart any special properties to the products. Citraconic anhydride is useful simply in producing liquid curing agents for epoxy resins. For example, Diels-Alder synthesis with isoprene yields a non-hygroscopic curing agent that is liquid at room temperature.

Therefore, it is a surprising and beneficial finding that citraconic acid can be used not only in the synthesis of polymers, but also as a medicament. In one aspect, the invention therefore encompasses the first medical indication of citraconic acid or derivatives thereof and its use as a medicament, independent of the particular disease setting, is a novel and unexpected finding that could not have been derived from the prior art.

In one embodiment, the invention relates to citraconic acid or derivatives thereof for use in the treatment of a medical condition associated with inflammation.

In other embodiments, the invention relates to citraconic acid or derivatives thereof as described herein for use in the treatment of inflammation. In embodiments, the citraconic acid or derivatives thereof has a direct effect on inflammation as a pharmaceutically active ingredient.

For example, the citraconic acid or derivatives thereof as described herein exhibit a direct effect on inflammation, which is considered distinct from its use as an acidifying component as such, as a carrier, as an excipient in generating a drug salt, or as an agent for improved solubility or adsorption of another pharmaceutically active agent. In embodiments, the use of citraconic acid or derivatives thereof as described herein as a medicament against inflammation relates to administration of the CA or derivative with the purpose of (directly) reducing (unwanted) inflammation in the treatment of a medical condition associated with inflammation. The prior art may for example teach CA or derivatives thereof being used as an excipient, carrier or solubility agent, without any mention of its effect in treating inflammation directly.

In embodiments, the invention relates to citraconic acid or derivatives thereof for use in treating inflammation in the context of any one or more medical conditions disclosed herein associated with inflammation.

Citraconic acid has been identified as an endogenous metabolite and is not detected in myeloid immune cells, but in the kidney, lymph nodes, and brain. It differs from itaconic acid in the location of the internal double bond and in the 3D structure, implying that it may have entirely different biochemical interactions and effects from itaconic acid. Therefore, it was not expected, with all these structural and functional differences, that citraconic acids inhibits the inflammatory response. More surprisingly, it inhibits inflammation and production of interferons more efficiently than itaconic acid. Furthermore, citraconic acid reduces reactive oxygen species (ROS) and phosphorylation of STAT1, thereby enabling the inhibition of inflammation and oxidative stress.

Citraconic acid is therefore similar in structure to both itaconic acid and mesaconic acid, but exhibits a structural difference that leads to distinct and improved biological properties. A skilled person could not have deduced from the prior art, or expected based on common knowledge, that the change in the internal double bond and thus in the 3D structure of the substance that an improvement in immunoregulation could have been achieved. To illustrate these differences, the structures of itaconic acid, mesaconic acid and citraconic acid are presented in more detail below, with the stereochemistry of the internal double bond illustrated.

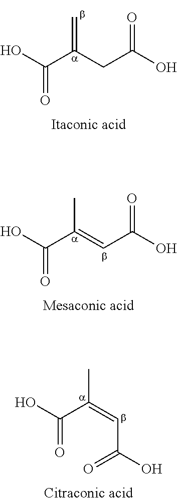

As has been shown by the inventors, in more detail below in the examples of the present application, citraconic acid exhibits multiple unexpected biological effects relevant in modulating inflammation and thus relevant in treating inflammation-related diseases. The biological effects of CA as outlined below are fundamental to multiple inflammatory process and thus relevant to treating various forms of inflammation.

Surprisingly, mitochondrial reactive oxygen species (ROS) are significantly decreased by citraconic acid, which is shown in the acute monocytic leukaemia cells infected with influenza A virus and incubated with citraconic acid. More surprisingly, the phosphorylation levels of STAT1 are reduced by citraconic acid, which is shown in adenocarcinoma human alveolar basal epithelial cells and acute monocytic leukaemia cells infected with influenza A virus and incubated with citraconic acid. The reduction of ROS enables the inhibition of inflammation and oxidative stress. This also indicates that citraconic acid may suppress viral genome replication and increase survival of infected cells.

In one embodiment, the invention relates to citraconic acid or derivative thereof for use in treating a medical condition, wherein treatment with citraconic acid or an ester thereof induces an Nrf2 anti-inflammatory signaling pathway. This surprising find is based on a quantum chemical study which shows citraconic acid is the strongest electrophile and Nrf2 agonist and mesaconic acid a weaker electrophile.

Itaconic acid has validated anti-inflammatory effects, but its efficacy is limited by very low electrophilicity and thus minimal induction of the cell protective Nrf2 pathway. The structurally modified substance dimethylitaconate can be used at lower doses, possibly due to a higher electrophilicity, but appears sub-optimal. It was surprising to find that the mRNA levels of Nrf2 are significantly increased by treatment with citraconic acid compared itaconic acid, indicating the inflammation supressing effect of citraconic acid.

In one embodiment, the invention relates to citraconic acid or derivative thereof for use in treating a medical condition, wherein treatment with citraconic acid or a derivative thereof inhibits synthesis of itaconic acid.

In one embodiment, the invention relates to citraconic acid or derivative thereof for use in treating a medical condition, wherein treatment with citraconic acid or a derivative thereof inhibits aconitate decarboxylase 1 (ACOD1) activity.

ACOD1-mediated itaconic acid production contributes to the antimicrobial activity of macrophages. Itaconic acid is also a growth factor for certain tumors. Inhibition of synthesis of itaconic acid can therefore be relevant for and used in cancer treatment. Currently, there is no directly acting ACOD1-inhibitor on the market. Surprisingly, the enzyme activity of ACOD1 is inhibited by citraconic acid which is shown below in a diagram showing inhibition of human cis-aconitate by citraconic acid. Human ACOD1 (hACOD1) activity was measured in the presence of increasing concentrations of substrate i.e. cis-aconitate and inhibitor i.e. citraconic acid.

In one embodiment, the invention relates to citraconic acid or derivative thereof for use in treating a medical condition, wherein treatment with citraconic acid or a derivative thereof inhibits expression and/or activity of an immune-response stimulating cytokine, such as an interferon, preferably CXCL10.

Furthermore, citraconic acid inhibits surprisingly the expression and/or activity of an immune response stimulating cytokine, such as an interferon, preferably CXCL10. This is shown by reduction of CXCL10 mRNA levels greatest by citraconic acid compared to itaconic acid and mesaconic acid indicating the anti-inflammatory effect of citraconic acid. This technical effect indicates that citraconic acid is effective in directly reducing inflammation, also by way of immunomodulation, thus is also relevant for reducing unwanted immune reactions that may lead to pathological inflammation. For example, autoimmune diseases, or any given disease with a recognized autoimmune component, may be susceptible to treatment with citraconic acid or derivatives thereof due to the shown efficacy against interferon production, in particular CXCL10.

In one embodiment, the invention relates to citraconic acid or derivative thereof for use in treating a medical condition, wherein treatment with citraconic acid or a derivative thereof exhibits an anti-oxidative effect (preferably reducing reactive oxygen species (ROS)).

Reactive oxygen species (ROS) are key signaling molecules that play an important role in the progression of inflammatory disorders. An enhanced ROS generation by polymorphonuclear neutrophils (PMNs) at the site of inflammation causes endothelial dysfunction and tissue injury. Under inflammatory conditions, oxidative stress produced by PMNs leads to the opening of inter-endothelial junctions and promotes the migration of inflammatory cells across the endothelial barrier. The migrated inflammatory cells not only help in the clearance of pathogens and foreign particles but also lead to tissue injury. The finding that citraconic acid also enables effective reduction in ROS is a surprising and beneficial aspect of citraconic acid or derivative thereof treatment, showing that unwanted immune responses causing inflammation and tissue damage may be countered by citraconic acid or derivative thereof, making it plausible that citraconic acid or derivatives thereof are applicable in a wide range of inflammatory disorders.

In one embodiment, the invention relates to citraconic acid or derivative thereof for use in treating a medical condition, wherein treatment with citraconic acid or a derivative thereof inhibits phosphorylation of signal transducer and activator of transcription 1 (STAT1).

Recent reports have shown that STAT1 rapidly undergoes phosphorylation following TLR4 challenge with lipopolysaccharide (LPS) in a model of LPS hypersensitivity in vivo. Furthermore, genetic ablation of STAT1 protected against LPS-induced lethality, suggesting that STAT1 plays a key role in Toll-like receptor-induced inflammation. Furthermore, Most neurological diseases are associated with chronic inflammation initiated by the activation of microglia, which produce cytotoxic and inflammatory factors. Signal transducers and activators of transcription (such as STAT1) are potent regulators of gene expression, thus STAT-induced inflammatory gene expression and STAT-dependent transcriptional networks appear to underlie brain inflammation. The reduction of STAT1 phosphorylation by citraconic acid therefore represents an unexpected and beneficial technical effect, indicating the suitability of the compound to treat various forms of inflammation, in particular those associated with STAT1 transcriptional regulation. The invention therefore also relates to the treatment of medical conditions comprising inflammation induced or associated with STAT1-phosphorylation. A skilled person is capable of identifying such diseases based on common knowledge and routine approaches towards investigating STAT1 activity and gene regulation in any given inflammatory disease.

In one embodiment, the treatment with citraconic acid or a derivative thereof interacts with the nuclear export factor chromosome region maintenance 1 (CRM1), also known as exportin 1 (XPO1), wherein preferably Cys528 in the nucleoprotein-binding groove of CRM1 is targeted.

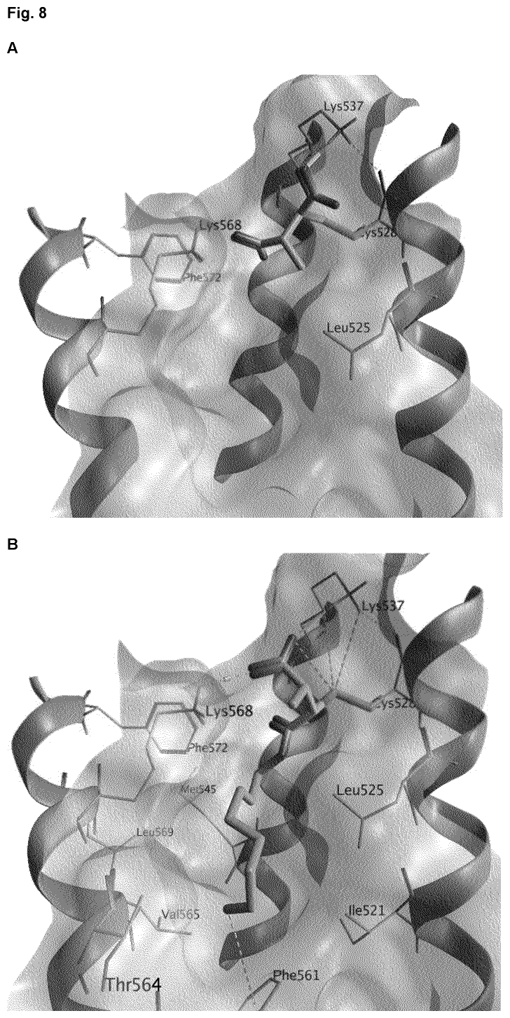

A docking study of citraconic acid and derivatives thereof into CRM1, which is an anti-viral target, showed citraconate, mesaconate, itaconate and 4-octyl citraconate efficiently undergo Michael 1,4-addition reaction with the Cys528 residue of CRM1 (Table 1 and FIG. 8). Both covalently bound compounds establish hydrogen bonds between their free carboxyl groups and Lys537 and Lys568. Surprisingly, 4-octyl citraconate shows more favourable binding through hydrophobic interactions between the octyl chain and IIe521, Leu525, Met545, Phe561, Val565 and Leu569 in the nucleoprotein-binding site.

One advantage derived from this surprising finding is that CA or a derivative thereof, or its isomer mesaconate or a derivative thereof, can be used for an anti-viral treatment, more specifically, for treating a subject who can benefit from blocking the XPO1 (CRM1) pathway. This would apply to patients infected with any virus that depends on XPO1 activity for part of its life cycle and is thus not limited to influenzaviruses but also includes coronaviruses, respiratory syncytial viruses and others.

In one embodiment, the medical condition associated with inflammation comprises chronic inflammation.

Preliminary studies indicate that citraconic acid or derivatives thereof may exhibit a low toxicity, thus these substances are considered suitable for long-term or recurring treatment of chronic unwanted inflammation. Doses and administration regimes can be developed by a skilled person accordingly. Preferred but non-limiting examples of diseases comprising chronic inflammation are cardiovascular disease, cancer, allergy, obesity, diabetes, digestive system diseases, degenerative diseases, autoimmune disorders, rheumatoid arthritis and neurodegenerative disease. In some embodiments, the medical condition associated with chronic inflammation is selected from the group consisting of cardiovascular disease, diabetes, autoimmune disease, rheumatic disease, allergy, arthritis, bowel diseases, psoriasis, pulmonary disease, graft vs host disease (GVHD) and transplant rejection.

In one embodiment, the medical condition associated with inflammation comprises acute inflammation.

Preliminary studies indicate that citraconic acid or derivative thereof directly act on inflammation pathways involved in multiple inflammatory conditions, thus these substances are considered suitable for treatment of conditions with acute inflammation. Doses and administration regimes can be developed by a skilled person accordingly.

Surprisingly, CXCL10 mRNA which encodes pro-inflammatory chemokine IP-10, is reduced by citraconic acid significantly more than itaconic acid and mesaconic acid in the human macrophage-like cells that are infected with influenza A virus.

In one embodiment, the medical condition associated with inflammation comprises immune paralysis. This is a surprising finding that CA or derivative thereof is capable of not only reducing the inflammation but also activating the immune system, i.e. modulating the immune system. In one embodiment, the acute inflammatory condition is a lung inflammation.

In one embodiment, the medical condition associated with inflammation is a viral infection, preferably an influenza virus, zika virus or coronavirus infection.

In one embodiment, the influenza virus is an influenza A virus.

It is a surprising finding that citraconic acid inhibits the production of influenza A progeny virions without affecting viral RNA synthesis. This surprising finding provides further support for the anti-viral effect of citraconic acid and derivative thereof.

In one embodiment, the coronavirus is a SARS-COV virus, such as SARS-COV-2.

In one embodiment, treatment with citraconic acid or derivative thereof inhibits replication of the viral genome.

It is known in the art that the antiviral activity of itaconic acid is mediated by inhibiting cellular succinate dehydrogenase (SDH), which leads to a metabolic state that suppresses viral genome replication. However, preliminary data demonstrates that citraconic acid does not directly bind SDH, thus indicating that distinct mechanisms are responsible for the anti-viral effect. Nevertheless, citraconic acid appears responsible in directly inhibiting viral replication, thus acting in a beneficial manner to influence two pathological causes of viral disease, by both inhibiting viral replication as such and by reducing an unwanted inflammation in response to viral infection. There is no suggestion in the art that citraconic acid or derivatives thereof may have been involved in this manner.

In one embodiment, the lung (pulmonary) disease is COVID-19. SARS-COV-2 infection and lung cell destruction triggers a local immune response that recruits macrophages and monocytes that respond to infection, release cytokines, and trigger adaptive T-and B-cell immune responses. In some cases, a dysfunctional immune response occurs that can cause severe pulmonary and even systemic pathologies. Therefore, controlling the inflammatory response by citraconic acid or derivative thereof represents an advantage in the treatment of COVID-19.

In one embodiment, the invention relates to citraconic acid or derivative thereof for use in treating a medical condition, wherein the medical condition is associated with inflammation and is a severe acute inflammatory syndrome, for example caused by an infection, such as a severe acute respiratory syndrome (SARS), sepsis, septic shock, multiple organ failure or a cytokine release syndrome.

In one embodiment, the medical condition to be treated is fever.

Fever, also referred to as pyrexia, is defined as having a temperature above the normal range due to an increase in the body's temperature set point. There is not a single agreed-upon upper limit for normal temperature with sources using values between 37.2 and 38.3 deg C in humans. A fever can be caused by many medical conditions ranging from non-serious to life-threatening. This includes viral, bacterial, and parasitic infections, such as influenza, the common cold, meningitis, urinary tract infections, appendicitis, COVID-19, and malaria. Non-infectious causes include vasculitis, deep vein thrombosis, connective tissue disease, side effects of medication, and cancer. Treatment to reduce fever is as such at times necessary although not always required. Nevertheless, treatment of the associated pain and inflammation, however, may be useful and help a person who is suffering from fever. Medications such as ibuprofen or paracetamol are known to lower temperature of subject. Citraconic acid or derivatives thereof therefore represent a promising class of compounds in reducing fever, reducing inflammation and associated pain in subjects, in an analogous clinical setting to known fever-reducing compounds.

In one embodiment, the medical condition to be treated is pain.

The inflammatory response typically represents a series of orchestrated physiological processes that occur after injury or infection in an attempt to combat and resolve the pathology. It is characterized by five classic symptoms: redness (rubor), heat (calor), swelling (tumor), pain or hypersensitivity (dolor), and loss of function (functio lasea). Under normal conditions, inflammation is an important protective mechanism essential for wound healing. Despite this, acute inflammation produces overt pain through the direct activation of sensory neurons that conduct the pain signal, and unwanted inflammation, such as in autoimmune conditions, lead to painful unnecessary immune responses caused to some extent by the underlying inflammation. Citraconic acid or derivatives thereof therefore represent a promising class of compounds in reducing pain and reducing associated inflammation in subjects, in an analogous clinical setting to known pain-reducing compounds.

In one embodiment, the medical condition to be treated is a tumor disease.

Tumors and inflammation exhibit a complex relationship, whereby in various tumor entities an anti-inflammatory approach to treatment may be beneficial To some extent, immune responses against tumors need to be enhanced, for example in modern immune therapies such as antibody or T cell therapy of cancers. Nevertheless, inflammation also drives tumor development and can be effectively reduced in tumor therapy approaches. Inflammation predisposes to the development of cancer and promotes all stages of tumorigenesis. Cancer cells, as well as surrounding stromal and inflammatory cells, engage in well-orchestrated reciprocal interactions to form an inflammatory tumor microenvironment (TME). Cells within the TME are highly plastic, continuously changing their phenotypic and functional characteristics. Various mechanisms show that inflammation drives tumor initiation, growth, progression, and metastasis.

One of the most promising recent developments in the field of cancer immunology is the successful implementation of various cancer immunotherapies, which use various approaches to redirect or hyperactivate the immune system toward the recognition, restraining, and killing of cancer cells. These approaches include immunological “checkpoint” blockade, immunization with cancer vaccines, neutralization of immunosuppressive cells, treatment with oncolytic viruses, or employing synthetic biology with bi-specific antibodies or cells with “chimeric antigen receptors” (CARs). However, the immune system plays a distinct role during tumor initiation, promotion, and progression, which is often referred as “cancer-promoting inflammation”. Although tumors might initially be characterized by T cell infiltration or their functional activation, such tumors still might present with upregulation of inflammatory mediators and recruitment of other immune cells, often with tumor-promoting properties, for example macrophages, monocytes, neutrophils or innate lymphoid cells (ILCs). Recent epidemiological studies implicate inflammation and tissue repair immune responses to enhanced tumor incidence, growth, and progression (Greten et al, Immunity, 51:27-41, 2019). Thus citraconic acid represents a promising approach towards immune regulation, useful in treating tumor disease.

In embodiments, citraconic acid or derivative thereof exhibits antineoplastic effect in tumor types whose growth requires or is otherwise dependent on ACOD1 activity, which is achieved e.g., either by ACOD1 overexpression in the tumor itself or by release of itaconate by neighboring cells (e.g., macrophages). Embodiments of the invention therefore relate to the treatment of cancers characterized by ACOD1 overexpression and/or dependence for cell growth on ACOD1 activity, which can be determined by a skilled person using established means.

In one embodiment, the tumor disease, or fever or any medical condition usually associated with inflammation, which may ultimately not be related to inflammation or in which inflammation is not a primarily pathological factor, can also be subjected to the treatment with citraconic acid or derivative thereof. In some embodiments, citraconic acid or derivatives thereof can directly treat tumor diseases or fever or viral infection without immune modulation being the primary therapeutic component, citraconic acid and derivatives thereof can in some embodiments exhibit direct anti-tumor, anti-viral, or anti-fever effects, in some cases independently of a reduction in inflammation.

In one embodiment, citraconic acid or derivative thereof is used for treating neurodegenerative diseases. Citraconic acid can be used to reduce chronic neuroinflammation or oxidative stress and to reduce endogenous synthesis of itaconic acid, as the latter may have neurotoxic effects (Umbrasas 2021, J Bioenerg Biomembr. 2021 October; 53 (5): 499-511)

Therefore, in some embodiments, the invention relates to citraconic acid or derivatives thereof for use as described herein in the treatment of a medical condition not associated with inflammation, or a medical condition comprising inflammation, but in which inflammation is not a primary pathological component, such as in some cases a tumor disease or fever. In most cases, tumor diseases and fever are associated with inflammation. Nevertheless, in some situations, if the tumor disease or fever is not diagnosed with a primary inflammatory component, or initially not diagnosed relating to inflammation, they still fall into the scope of treatment with citraconic acid or derivatives thereof as described herein.

In one aspect, the invention relates to citraconic acid or derivative thereof according to formula VII

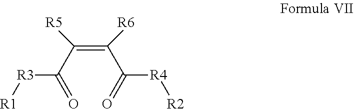

wherein R1 and R2 are, independently, H, a branched or straight chain alkyl, alkenyl, alkynyl, aryl, cycloalkyl or alkoxy group of C1-C12, preferably C1-C8, more preferably C1-C8 alkyl, wherein R1 and/or R2 is optionally substituted with halogen, alkoxy, hydroxy, amine or C1-C8 alkyl,
wherein R3 and R4 are, independently, a heteroatom, preferably O, S or N.
wherein R5 and R6 are independently, H, a branched or straight chain alkyl, alkenyl, alkynyl, aryl, heteroaryl, cycloalkyl or alkoxy group of C1-C12, preferably C1-C8, more preferably C1-C4 alkyl or phenyl, wherein R5 and/or R6 is optionally substituted with halogen, alkoxy, hydroxy, amine or C1-C8 alkyl,
wherein preferably R6 is H,
wherein optionally, R5 is fused to R6 to form a cyclic structure, preferably a 5-or 6-membered structure, preferably a cyclopentene.

In one embodiment, the compound is defined by at least one of the following features:
when R3 and R4 are O, at least one of R1 or R2 is not H,
R5 is aryl, preferably phenyl, and/or
R5 is fused to R6 to form a cyclic structure, preferably a 5- or 6-membered structure, preferably a cyclopentene.

In one embodiment, the Z stereo confirmation is preferred, as shown by way of example in Formulae XI and XII, for any one or more of the formulae disclosed herein.

In one embodiment, when R3 and R4 are O, at least one of R1 and R2 is not H. This embodiment represents citraconic acid derivatives, similar to Formula I, excluding citraconic acid itself.

In one embodiment, R3 and R4 are both O.

In one embodiment, R3 and R4 are, independently, O, S or N.

In one embodiment, R3 and R4 are both O, R5 is alkyl, preferably a straight alkyl chain, more preferably ethyl, and R6 is H.

In one embodiment, R3 and R4 are both O, R5 is phenyl, and R6 is H.

In one embodiment, R3 and R4 are both O, and R5 is fused to R6 to form a cyclic structure.

In one embodiment, R3 and R4 are both O, and R5 is fused to R6 to form cyclopentene.

The fusing of R5 and R6 may be understood as a bond between R5 and R6, or as any number of bonds and/or groups positioned between R5 and R6, thus optionally linking R5 and R6 via another group. For example, in some embodiments, a —CH2—alkyl group may be positioned between R5 and R6, forming a 5-membered ring structure comprising R5 and R6. In other embodiments, a —CH2—CH2— alkyl group may be positioned between R5 and R6, forming a 6-membered ring structure comprising R5 and R6. Fusion of R5 to R6, forming a cyclic structure, may be via any given bond or group chemically compatible to form the cyclic structure.

In one embodiment, R5 is fused to R6 to form a 5-membered cyclic structure, preferably a cyclopentene, wherein the cyclic structure comprising R5 and R6 is optionally substituted with C1-C8 alkyl, preferably c1 alkyl.

In one embodiment, the cyclic structure comprising R5 and R6 is optionally substituted with halogen, alkoxy, hydroxy, amine or C1-C8 alkyl, preferably C1 alkyl.

In one embodiment, the cyclic structure comprising R5 and R6 is not substituted with halogen, alkoxy, hydroxy, amine or C1-C8 alkyl.

In embodiments, a compound of Formula VII is for use in the treatment of one or more of the medical conditions disclosed herein.

In a preferred embodiment, R3 and R4 are both O, R1, R2 and R6 are H, according to formula XI

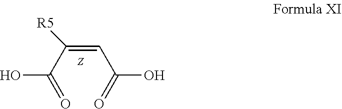

In another preferred embodiment, R3 and R4 are both O, R1 and R2 are both H, according to formula XII.

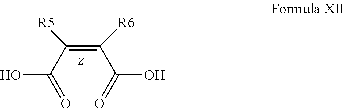

In one aspect, the invention relates to citraconic acid or derivative thereof according to formula I

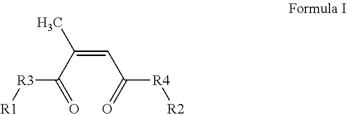

wherein R1 and R2 are, independently, H, a branched or straight chain alkyl, alkenyl, alkynyl, aryl, cycloalkyl or alkoxy group of C1-C12, preferably C1-C8, more preferably C1-C8 alkyl, wherein R1 and/or R2 is optionally substituted with halogen, alkoxy, hydroxy, amine or C1-C8 alkyl,
wherein R3 and R4 are, independently, a heteroatom, preferably O, S or N.

In one embodiment, when R3 and R4 are O, at least one of R1 and R2 is not H. This embodiment represents citraconic acid derivatives, which fall under Formula I, excluding citraconic acid itself.

In one embodiment, R3 and R4 are both O. This embodiment relates to a double ester derivative of citraconic acid.

In one embodiment, R3 and R4 are, independently, O, S or N.

In one embodiment, the citraconic acid derivative is an ester. For any given embodiment in which “citraconic acid or derivative thereof” is disclosed, said embodiment may be in other embodiments considered to encompass, and also to disclose, “citraconic acid or ester thereof”.

In embodiments, a compound of Formula I is for use in the treatment of one or more of the medical conditions disclosed herein.

In one embodiment, the citraconic acid derivative is an ester derivative according to formula II

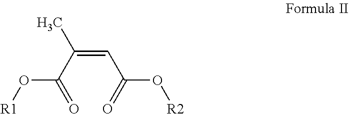

wherein R1 and R2 are, independently, a branched or straight chain alkyl, alkenyl, alkynyl, aryl, cycloalkyl or alkoxy group of C1-C12, preferably C1-C8, more preferably C1-C8 alkyl, wherein R1 and/or R2 is optionally substituted with halogen, alkoxy, hydroxy, amine or C1-C8 alkyl.

In one embodiment, of either formula I or II, R1 and R2 are, independently, a branched or straight chain alkyl, alkenyl, alkynyl, aryl or alkoxy group of C1-C12, preferably C1-C8, more preferably C1-C8 alkyl, wherein R1 and/or R2 is optionally substituted with halogen or C1-C8 alkyl.

In embodiments, the compound of Formula II is for use in the treatment of one or more of the medical conditions disclosed herein.

In a preferred embodiment, the invention relates to citraconic acid, according to formula III, wherein R3 and R4 of formula I are both O, and R1 and R2 are both H.

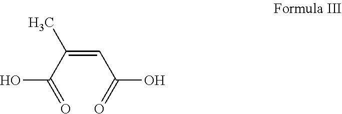

According to the knowledge of the inventors, the derivatives of citraconic acid described herein have not been described previously, thus the structures of these agents is novel, and the derivative compounds are considered to also exhibit the beneficial biological effects as for citraconic acid, as described herein. Thus, due to the central double bond and resulting 3D geometries of the compound core, citraconic acid and the derivatives thereof described herein show novel and unexpected biological effects, common to this series of compounds.

In embodiments, a compound of Formula III is for use in the treatment of one or more of the medical conditions disclosed herein.

In some embodiments, the citraconic acid derivative as described herein is according to formula IV (WAM474), formula V (WAM475) or formula VI (WAM476):

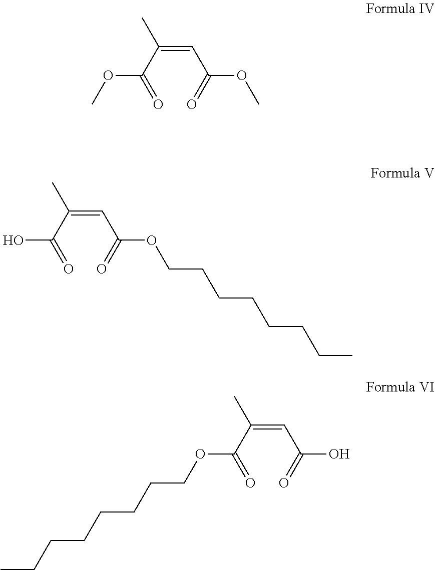

In embodiments, the citraconic acid derivative described herein is according to formula VIII ((Z)-2-ethylbut-2-enedioic acid) also called 2-Ethylmaleic acid or Methylcitraconic acid, formula IX (2-Phenylmaleic acid) or formula X (Cyclopent-1-ene-1,2-dicarboxylic acid).

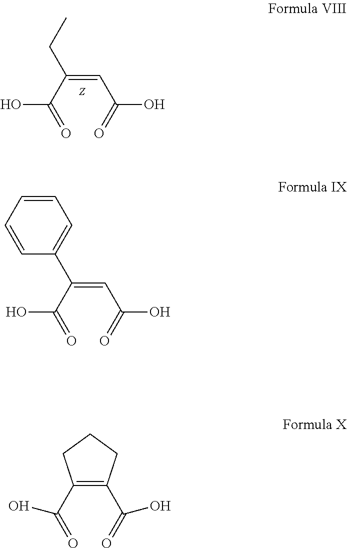

In embodiments, the citraconic acid derivative described herein is according to Formula XIII 3-Methyl-cyclopent-1-ene-1,2-dicarboxylic acid

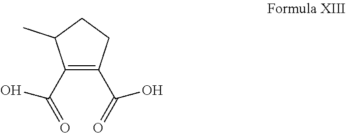

In embodiments, a compound of Formula IV, V, VI, VIII, IX, X and XIII is for use in the treatment of one or more of the medical conditions disclosed herein.

In another aspect, the invention relates to a pharmaceutical composition comprising citraconic acid or a derivative thereof, preferably those described herein, with one or more pharmaceutical excipients for use according as a medicament, in particular in the treatment of one or more diseases as described herein.

Advantageously, based on the surprising findings as described above, a pharmaceutical composition comprising citraconic acid or derivatives thereof, alone or with one or more pharmaceutical excipients, can be used to regulate various aspects of inflammation to reduce mortality in patients with tumor diseases, bacterial infection, viral infection, amongst others.

The features of the invention disclosed herein with respect to citraconic acid also relate to the derivative compounds described herein. Furthermore, the features of the invention disclosed herein with respect to citraconic acid or derivatives thereof also relate to the pharmaceutical composition, and vice versa.

### DETAILED DESCRIPTION OF THE INVENTION

All cited documents of the patent and non-patent literature are hereby incorporated by reference in their entirety.

### Citraconic Acid and Derivatives Thereof

Citraconic acid (synonym: 2-methylmaleic acid) is the cis stereoisomer of mesaconic acid and, like the similar itaconic acid, belongs to the isomeric dicarboxylic acids derived from citric acid.

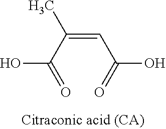

In some embodiments, citraconic acid derivatives according to formulae I-VI are covered by the invention, as described above.

With respect to the chemical compounds described herein, the term “alkyl” refers to a branched or unbranched saturated hydrocarbon group of preferably 1 to 7 carbon atoms, such as methyl, ethyl, n-propyl, isopropyl, n-butyl, isobutyl, t-butyl, pentyl, hexyl, heptyl, and the like. Preferred alkyl groups have 1-12 carbon atoms, more preferably 1-10, 1-8, 1-6, 1-5, 1-4 or 1-3, 2 or 1 carbon atoms. Any one or more of the alkyl groups described herein may be “substituted alkyls”, wherein one or more hydrogen atoms are substituted with a substituent such as halogen, cycloalkyl, alkoxy, hydroxyl, aryl, or carboxyl.

The term “alkenyl” refers to a straight, branched or cyclic hydrocarbon configuration and combinations thereof, including preferably 2 to 12 carbon atoms, more preferably 2 to 8 carbon atoms, that would form if a hydrogen atom is removed from an alkene, for example resulting in ethenyl, or the like.

The term “alkynyl” refers a straight, branched or cyclic hydrocarbon configuration and combinations thereof, including preferably 2 to 12 carbon atoms, more preferably 2 to 8 carbon atoms, that would form if a hydrogen atom is removed from an alkyne, for example resulting in ethynyl, or the like.

The term “aryl” refers to any carbon-based aromatic group including, but not limited to, benzene, naphthalene, and the like. The term “aromatic” also includes a “heteroaryl group,” which is defined as an aromatic group that has at least one heteroatom incorporated within the ring of the aromatic group. Examples of heteroatoms include, but are not limited to, nitrogen, oxygen, sulfur. The aryl group can be substituted with one or more groups including, but not limited to, alkyl, aryl, halogen, nitro, hydroxy, carboxylic acid, or alkoxy, or the aryl group can be unsubstituted.

The term “cycloalkyl” refers to a configuration derived from a cycloalkane by removal of an atom of hydrogen, thereby forming preferably cyclopropyl, cyclobutyl, cyclopentyl, cyclohexyl or cycloheptyl, or the like.

The term “cyclic structure” refers to a structure wherein three or more atoms being connected to form a ring. The atoms can be only carbon or non-carbon, such as O, S, N or in the presence of carbon and non-carbon. Heterocyclic structures are included. The cyclic structure can be aromatic or non-aromatic.

The term “R5 fused to R6 to form a cyclic structure” refers to a resulting cyclic structure with at least three atoms, four atoms, five atoms, six atoms, or more, wherein preferred members of the cyclic structure are carbon atoms.

In one embodiment, R5 is fused to R6 to form an aromatic cyclic structure with 5 carbons or 6 carbons. In one embodiment, R5 is fused to R6 to form a cyclopentene.

The term “amine” refers to a group of the formula —NRR′, where R and R′ can be, independently, hydrogen or an alkyl, alkenyl, alkynyl, aryl, aralkyl, cycloalkyl, halogenated alkyl, or heterocycloalkyl group described above.

The term “hydroxyl” is represented by the formula —OH.

Optionally substituted groups, such as “optionally substituted alkyl,” refers to groups, such as an alkyl group, that when substituted, have from 1-5 substituents, typically 1, 2 or 3 substituents, selected from for example alkoxy, alkyl, aryl, halogen, hydroxyl. In particular, optionally substituted alkyl groups include, by way of example, haloalkyl groups, such as fluoroalkyl groups, including, without limitation, trifluoromethyl groups.

The term “alkoxy” refers to a straight, branched or cyclic hydrocarbon configuration and combinations thereof, including preferably 1-12 carbon atoms, more preferably 1-10, 1-8, 1-6, 1-5, 1-4 or 1-3 carbon atoms, that include an oxygen atom at the point of attachment (such as O-alkyl). An example of an “alkoxy group” is represented by the formula —OR, or —ROR, where R can be an alkyl group, optionally substituted with halogen, aryl, cycloalkyl, halogenated alkyl. Suitable alkoxy groups include methoxy, ethoxy, n-propoxy, i-propoxy, n-butoxy, i-butoxy, t-butoxy, cyclohexyloxy, and the like.

The term “heteroatom” refers to any atom that is not carbon or hydrogen, selected from the group of oxygen, nitrogen, sulfur, phosphrus, chlorine, bromine, iodine, lithium and magnesium. In a preferred embodiment, the heteroatom is oxygen, nitrogen or sulfur.

Protected derivatives of the disclosed compound also are contemplated, for example for use in the synthesis of the disclosed compounds. A variety of suitable protecting groups for use with the disclosed compounds are disclosed in Greene and Wuts Protective Groups in Organic Synthesis; 3rd Ed.; John Wiley & Sons, New York, 1999. In general, protecting groups are removed under conditions which will not affect the remaining portion of the molecule. These methods are well known in the art and include acid hydrolysis, hydrogenolysis and the like.

Particular examples of the presently disclosed compounds include one or more asymmetric centers; thus these compounds can exist in different stereoisomeric forms. Accordingly, compounds and compositions may be provided as individual pure enantiomers or as stereoisomeric mixtures, including racemic mixtures.

The compounds of the invention may also exist in various polymorphous forms, for example as amorphous and crystalline polymorphous forms. All polymorphous forms of the compounds of the invention belong within the framework of the invention and are a further aspect of the invention.

The compound of the invention may also comprise deuterium replacing hydrogen. This replacement may in some circumstances lead to improved metabolic stability (Nature Reviews Drug Discovery 15, 219-221 (2016)).

It is understood that substituents and substitution patterns of the compounds described herein can be selected by one of ordinary skill in the art to provide compounds that are chemically stable and that can be readily synthesized by techniques known in the art and further by the methods set forth in this disclosure.

The present invention relates further to pharmaceutically acceptable salts of the compounds described herein. The term “pharmaceutically acceptable salt” refers to salts or derivatives of the compounds described herein prepared by conventional means that include basic salts of inorganic and organic acids. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

### Medical Condition Associated with Inflammation

As used herein the term “medical condition associated with inflammation” refers to any medical condition associated with inflammation. Inflammation typically represents a series of orchestrated physiological processes that occur after injury or infection in an attempt to combat and resolve the pathology. It is characterized by one or more of five classic symptoms: redness (rubor), heat (calor), swelling (tumor), pain or hypersensitivity (dolor), and loss of function (functio lasea). According to the invention, the inflammation to be treated is preferably pathological, i.e. plays a role in the cause of the disease, but may also be a result or symptom of a disease to be treated. In some embodiments, the medical condition comprises an inflammatory response to a foreign body, such as a thorn, an irritant or a pathogen, such as bacteria, viruses, and other organisms or caused by a wide range of diseases selected but not limited to the group of autoimmune diseases, metabolic syndrome, tumor diseases. Medical conditions associated with inflammation can also be resulted from use of a medicament during the treatment of disease.

Inflammation is a complex process that occurs for example when tissues are infected or injured by harmful stimuli such as pathogens, damage, or irritants, or when an unwanted autoimmune reaction is instigated. Immune cells, blood vessels, and molecular mediators are involved in this protective response. Inflammation is also a pathological phenomenon associated with a variety of disease states induced mainly by physical, chemical, biological, and psychological factors. The aim of inflammation is to limit and eliminate the causes of cellular damage, clear and/or absorb necrotic cells and tissues, and initiate tissue repair.

Two forms of inflammation are typically identified: acute and chronic. Acute inflammation is self-limiting and beneficial to the host, but prolonged chronic inflammation is a common feature of many chronic diseases and complications. Direct infiltration by many mononuclear immune cells such as monocytes, macrophages, lymphocytes, and plasma cells, as well as the production of inflammatory cytokines, lead to chronic inflammation. It is recognized that chronic inflammation plays a critical role in carcinogenesis. In general, both pro-and anti-inflammatory signaling pathways interact in the normal inflammatory process.

As used herein “chronic inflammation” is considered any inflammatory disorder in which chronic inflammation is evident. Chronic inflammation can be identified by, and/or is mediated by mononuclear cells such as monocytes and lymphocytes. Chronic inflammation can be characterized as the simultaneous destruction and healing of tissue from the inflammatory process, the net result of which is to promote damage rather than mediate repair. Since inflammatory responses can occur anywhere in the body, chronic inflammation is associated with the pathophysiology of a wide range of seemingly unrelated disorders that underlie a wide variety of human diseases. Chronic inflammation has been implicated in various diseases such as cardiovascular disease, cancer, allergy, obesity, diabetes, digestive system diseases, degenerative diseases, autoimmune disorders, and Alzheimer's disease.

In some embodiments, a person may not fully recover from acute inflammation which may lead to chronic inflammation. Factors that may increase the risk of chronic inflammation include but not limited to older age, obesity, a diet that is rich in unhealthful fats and added sugar, smoking, low sex hormones, stress, or sleep problems. Long-term diseases that doctors associate with inflammation including asthma, chronic peptic ulcer, tuberculosis, rheumatoid arthritis, periodontitis, ulcerative colitis and Crohn's disease, sinusitis, active hepatitis. Inflammation plays a vital role in healing, but chronic inflammation may increase the risk of various diseases, including some cancers, rheumatoid arthritis, atherosclerosis, periodontitis, and hay fever.

As used herein, “acute inflammation” refers to the body's first protective inflammatory response, as commonly understood by skilled person. An acute inflammation typically removes damaging stimuli by maintaining tissue integrity and contributing to tissue repair. It is part of the body's natural defence system against injury and disease, and without acute inflammation, wounds and infections will never heal and progressive destruction of the tissue will jeopardize the organism's survival.

The process of acute inflammation is initiated in all tissues by pre-existing cells, which are mainly resident macrophages, dendritic cells, histiocytes, Kupffer cells, mast cells, vascular endothelial cells, and Vascular smooth muscle cells. When noxious stimuli are initiated, these cells are activated and become proinflammatory and inflammatory sensitizing molecules such as pro-inflammatory cytokines, pro-inflammatory prostaglandins, leukotrienes, histamine, serotonin, neutral proteases, bradykinins, and releases nitric oxide and the like. These inflammatory molecules regulate a series of complex biological events involving the cellular and non-cellular components of the local vasculature, immune system, and damaged tissue sites to proliferate and mature the inflammatory response. These events contribute to the induction of an acute inflammatory response, which is typically characterized by: (1) Vasodilation that causes erythema (redness and warmth) by increasing blood flow to the tissue. This can spread beyond the site (flare reaction); (2) Vascular permeability causing edema (swelling) by increasing plasma leakage to the tissue; (3) Altering excitability of certain sensory nerves cause hypersensitivity and pain; (4) peripheral nerves of pro-inflammatory molecules such as neuropeptides such as substance P (SP) and calcitonin gene-related peptide (CGRP), prostaglandins, and amino acids such as glutamate Stimulates release from the terminal; (5) increased migration of leukocytes, mainly granulocytes, from blood vessels to tissues. Acute inflammatory reactions require constant stimulation to be maintained and must be actively terminated when no longer needed. Thus, acute inflammation typically disappears once the damaging stimulus is removed.

As used herein, “immune paralysis” means a suppression of the immune system. A suppression of the immune system is, by way of example, produced in a subject by a stimulus received in such an amount that a subsequent immune activation (such as an immunization, or typically immune response activating agent) fails to stimulate immunity. In one embodiment, the treatment of citraconic acid or derivative thereof can modulate a paralysed immune system, so that an adequate immune response is enabled. In one embodiment, the stimulus is a viral antigen, for example a spike protein of a coronavirus.

As used herein “lung inflammation” shall mean inflammation of lung tissue. Non-limiting examples refer to pneumonitis, which can be caused by radiation therapy of the chest, exposure to medications used during chemotherapy, the inhalation of debris, aspiration herbicides or fluorocarbons and some systemic diseases. The causes of pneumonitis include further viral infection, pneumonia, inhaling chemicals, such as sodium hydroxide, interstitial lung disease, sepsis, adverse reaction to medications, hypersensitivity to inhaled agents, inhalation of spores of some of species of mushroom, mercury exposure, smoking, overexposure to chlorine, bronchial obstruction, ascariasis, aspirin overdose, some antibiotics, chemotherapy drugs, exposure to some types of thermophilic actinomyces, mycobacteria, molds, avian proteins in bird feces and feather, whole body or chest radiation therapy used for cancer treatment. Pneumonitis can be classified into different specific subcategories including hypersensitivity pneumonitis, radiation pneumonitis, acute interstitial pneumonitis, and chemical pneumonitis.

In one embodiment, lung inflammation shall mean pneumonia, which is a type of pneumonitis. Pneumonia is a local infection that inflames the air sacs in one or both lungs. Pneumonia can range in seriousness from mild to life-threatening. A variety of organisms, including bacteria, viruses and fungi can cause pneumonia.

In a preferred embodiment, pneumonia refers to community-acquired pneumonia. Community-acquired pneumonia is the most common type of pneumonia. It occurs outside of hospitals or other health care facilities. It may be caused by bacteria, preferably streptococcus pneumoniae, bacterial-like organisms, preferably, mycoplasma pneumoniae, fungi, preferably fungi in soil or bird droppings, viruses such as coronavirus, preferably, SARS-COV-2.

In another preferred embodiment, pneumonia refers to hospital-acquired pneumonia. Some people acquire pneumonia during a hospital stay for another illness. Hospital-acquired pneumonia can be serious because the bacteria causing it may be more resistant to antibiotics and because the people who get it are already sick. People who are on breathing machines (ventilators), often used in intensive care units, are at higher risk of this type of pneumonia.

In another embodiment, pneumonia refers to aspiration pneumonia. Aspiration pneumonia occurs when you inhale food, drink, vomit or saliva into your lungs. Aspiration is more likely if something disturbs your normal gag reflex, such as a brain injury or swallowing problem, or excessive use of alcohol or drugs.

In some embodiment, medical condition associated with inflammation includes also symptoms such as fever, chills, fatigue, loss of energy, headaches, loss of appetite, muscle stiffness. In some embodiment, these symptoms which may not be associated with inflammation are also suitable for use of citraconic acid as described herein. In some embodiment, fever is not associated with inflammation. It however still can be subjected to treatment of using citraconic acid or derivative thereof.

It is commonly understood that the hypothalamus controls body temperature. In response to an infection, illness or some other cause, the hypothalamus may reset the body to a higher temperature. So fever is a symbol for something going on in the body. Fever may be resulted from in infections of the ear, lung, skin, throat, bladder or kidney, heat exhaustion, sunburn, side effects of medications, vaccines and immunizations, blood clots, autoimmune condition, cancer, hormone disorders, illegal drugs such as, amphetamines, cocaine.

As used herein, “sepsis” refers to a systemic response to infection. In one embodiment, sepsis may be seen as the combination of systemic inflammatory response syndrome with a confirmed infectious process or an infection. In some embodiment, sepsis may be characterized as clinical syndrome defined by the presence of both infection and a systemic inflammatory response (Levy M M et al. 2001 SCCM/ESICM/ACCP/ATS/SIS International Sepsis Definitions Conference. Crit Care Med. 2003 April; 31 (4): 1250-6). The term “sepsis” used herein also includes, but is not limited to, sepsis, severe sepsis, septic shock.

The term “sepsis” used herein includes, but is not limited to, sepsis, severe sepsis, and septic shock. Severe sepsis in refers to sepsis associated with organ dysfunction, hypoperfusion abnormality, or sepsis-induced hypotension. Hypoperfusion abnormalities include lactic acidosis, oliguria and acute alteration of mental status. Sepsis-induced hypotension is defined by the presence of a systolic blood pressure of less than about 90 mm Hg or its reduction by about 40 mm Hg or more from baseline in the absence of other causes for hypotension (e.g. cardiogenic shock). Septic shock is defined as severe sepsis with sepsis-induced hypotension persisting despite adequate fluid resuscitation, along with the presence of hypoperfusion abnormalities or organ dysfunction (Bone et al., CHEST 101 (6): 1644-55, 1992).

The term sepsis may alternatively be defined as life-threatening organ dysfunction caused by a dysregulated host response to infection. For clinical operationalization, organ dysfunction can preferably be represented by an increase in the Sequential Organ Failure Assessment (SOFA) score of 2 points or more, which is associated with an in-hospital mortality greater than 10%. Septic shock may be defined as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia.

The term “sepsis” used herein relates to all possible stages in the development of sepsis. The term “sepsis” also includes severe sepsis or septic shock based on the SEPSIS-2 definition (Bone et al., 2009). The term “sepsis” also includes subjects falling within the SEPSIS-3 definition (Singer et al., 2016). The term “sepsis” used herein relates to all possible stages in the development of sepsis.

As used herein, “multiple organ failure” (MOF), also known as multiple organ dysfunction syndrome (MODs), refers to altered organ function in an acutely ill patient such that homeostasis cannot be maintained without medical intervention. It is well established that Systemic Inflammatory Response Syndrome (SIRS) will lead to sepsis or severe sepsis and eventually lead to MODS. MODS usually results from uncontrolled inflammatory response which is triggered by infection, injury (accident or surgery), hypoperfusion and/or hypermetabolism. The uncontrolled inflammatory response will lead to SIRS or sepsis.

SIRS is an inflammatory state affecting the whole body. It is one of several conditions related to systemic inflammation, organ dysfunction, and organ failure. SIRS is a subset of cytokine storm, in which there is abnormal regulation of various cytokines. The cause of SIRS can be classified as infectious or non-infectious. SIRS is also closely related to sepsis. When SIRS is due to an infection, it is considered as sepsis. Non-infectious causes of SIRS include trauma, burns, pancreatitis, ischemia and haemorrhage. Sepsis is a serious medical condition characterized by a whole-body inflammatory state. Sepsis can lead to septic shock, multiple organ dysfunction syndrome and death. Both SIRS and sepsis could ultimately progress to MODS.

As used herein, “cytokine release syndrome (CRS)” refers to is a form of systemic inflammatory response syndrome (SIRS) that can be triggered by a variety of factors such as infections and certain drugs. In some embodiment, it refers to cytokine storm syndromes (CSS) and occurs when large numbers of white blood cells are activated and release inflammatory cytokines, which in turn activate yet more white blood cells. CRS is also an adverse effect of some monoclonal antibody medications, as well as adoptive T-cell therapies. When occurring as a result of a medication, it is also known as an infusion reaction.

CRS occurs when large numbers of white blood cells, including B cells, T cells, natural killer cells, macrophages, dendritic cells, and monocytes are activated and release inflammatory cytokines, which activate more white blood cells in a positive feedback loop of pathogenic inflammation (Lee D W, Gardner R, Porter D L, Louis C U, Ahmed N, Jensen M, et al. (July 2014). “Current concepts in the diagnosis and management of cytokine release syndrome”. Blood. 124 (2): 188-95.). Immune cells are activated by stressed or infected cells through receptor-ligand interactions (Liu Q, Zhou Y H, Yang Z Q (January 2016). “The cytokine storm of severe influenza and development of immunomodulatory therapy”. Cellular & Molecular Immunology. 13 (1): 3-10).

This can occur when the immune system is fighting pathogens, as cytokines produced by immune cells recruit more effector immune cells such as T-cells and inflammatory monocytes (which differentiate into macrophages) to the site of inflammation or infection. In addition, pro-inflammatory cytokines binding their cognate receptor on immune cells results in activation and stimulation of further cytokine production (Murphy K, Travers P, Walport M (2007). “Signaling Through Immune System Receptors”. Janeway's Immunobiology (7th ed.). London: Garland.). This process, when dysregulated, can be life-threatening due to systemic hyper-inflammation, hypotensive shock, and multi-organ failure.

In some embodiments, the medical condition associated condition is a tumor disease. Inflammation is a critical component of tumor progression. Many cancers arise from sites of infection, chronic irritation and inflammation. It is now becoming clear that the tumor microenvironment, which is largely orchestrated by inflammatory cells, is an indispensable participant in the neoplastic process, fostering proliferation, survival and migration. In addition, tumor cells have co-opted some of the signaling molecules of the innate immune system, such as selectins, chemokines and their receptors for invasion, migration and metastasis.

Cytokine and chemokine balances regulate neoplastic outcome. The balance of cytokines in any given tumor is critical for regulating the type and extent of inflammatory infiltrate that forms. Tumors that produce little or no cytokines or an overabundance of anti-inflammatory cytokines induce limited inflammatory and vascular responses, resulting in constrained tumor growth. In contrast, production of an abundance of pro-inflammatory cytokines can lead to a level of inflammation that potentiates angiogenesis, thus favouring neoplastic growth. Alternatively, high levels of monocytes and/or neutrophil infiltration, in response to an altered balance of pro-versus anti-inflammatory cytokines, can be associated with cytotoxicity, angiostasis and tumor regression. In tumors, interleukin-10 is generally a product of tumor cells and tumor-associated macrophages.

As used herein, the term “neurodegenerative disease” shall mean a condition which primarily affect reduced function of neurons in the central nervous system, preferably in the brain. It includes but is not limited to dementia, Alzheimer's disease, Parkinson's disease, prion disease, motor neuron disease, Huntington's disease, frontotemporal dementia, spinocerebellar ataxia, spinal muscular atrophy.

### Anti-Viral Effect

As used herein, “viral infection” occurs when an organism's body is invaded by a pathogenic virus. The virus type is preferably selected from the group consisting of adenovirus, coxsackievirus, Epstein-barr virus, Hepatitis A virus, hepatitis B virus, hepatitis C virus, Herpes simplex virus type 1, herpes simplex virus type 2, cytomegalovirus, human herpesvirus type 8, HIV, influenza virus, measles virus, mumps virus, human papillomavirus, poliovirus, rabies virus, respiratory syncytial virus, rubella virus, varicella-zoster virus, SARS-COV-2 virus. In one embodiment, the family of the virus is preferably selected from the group of Adenoviridae, Picornaviridae, Herpesviridae, Picornaviridae, Hepadnaviridae, Flaviviridae, Herpesviridae, Retroviridae, Orthomyxoviridae, Paramyxoviridae, Papillomavirus, Picornaviridae, Rhaboviridae, Togaviridae, Herpesviridae and Coronaviridae.

As used herein “influenza virus”, an enveloped RNA virus belonging to Orthomyxoviridae family.

Influenza viruses first cause infection in human by infecting epithelial cells in the respiratory tract. Illness during infection is primarily the result of lung inflammation and compromise caused by epithelial cell infection and death, combined with inflammation caused by the immune system's response to infection. Non-respiratory organs can become involved. Severe respiratory illness can be caused by multiple, non-exclusive mechanisms, including obstruction of the airways, loss of alveolar structure, loss of lung epithelial integrity due to epithelial cell infection and death, and degradation of the extracellular matrix that maintains lung structure. In particular, alveolar cell infection appears to drive severe symptoms since this results in impaired gas exchange and enables viruses to infect endothelial cells, which produce large quantities of pro-inflammatory cytokines.

Pneumonia caused by influenza viruses is characterized by high levels of viral replication in the lower respiratory tract, accompanied by a strong pro-inflammatory response called a cytokine storm. Infection with H5N1 or H7N9 especially produces high levels of pro-inflammatory cytokines. In bacterial infections, early depletion of macrophages during influenza creates a favourable environment in the lungs for bacterial growth since these white blood cells are important in responding to bacterial infection. Host mechanisms to encourage tissue repair may inadvertently allow bacterial infection. Infection also induces production of systemic glucocorticoids that can reduce inflammation to preserve tissue integrity but allow increased bacterial growth.

Cells possess sensors to detect viral RNA, which can then induce interferon production. Interferons mediate expression of antiviral proteins and proteins that recruit immune cells to the infection site, and they also notify nearby uninfected cells of infection. Some infected cells release pro-inflammatory cytokines that recruit immune cells to the site of infection. Immune cells control viral infection by killing infected cells and phagocytizing viral particles and apoptotic cells. An exacerbated immune response, however, can harm the host organism through a cytokine storm. To counter the immune response, influenza viruses encode various non-structural proteins, including NS1, NEP, PB1-F2, and PA-X, that are involved in curtailing the host immune response by suppressing interferon production and host gene expression. Influenza virus has four types: Influenza A virus, Influenza B virus, Influenza C virus and influenza D virus.

In one embodiment, viral infection refers to infection of virus family Flaviviridae including Zika virus, dengue virus, yellow fever virus, Japanese encephalitis and West Nile viruses. They are enveloped positive-strand RNA virus which mainly infect mammals and birds.

In some embodiments, viral infection refers to infection of zika virus. The infection of Zika virus causes no or only mild symptoms, similar to a very mild form of dengue fever. While there is no specific treatment so far available. Zika virus can spread from a pregnant woman to her baby resulting in microcephaly, severe brain malformations and other birth defects. Zika infections in adults may result rarely in Guillain-Barre syndrome. Symptoms of Zika virus infection includes fever, red eyes, joint pain, headache and a maculopapular rash.

In one embodiment, the viral infection is by a coronavirus. Coronaviruses are a group of related viruses that cause diseases in mammals and birds. The scientific name for coronavirus is Orthocoronavirinae or Coronavirinae. Coronavirus belongs to the family of Coronaviridae. The family is divided into Coronavirinae and Torovirinae sub-families, which are further divided into six genera: Alphacoronavirus, Betacoronavirus, Gammacoronavirus, Deltacoronavirus, Torovirus, and Bafinivirus. While viruses in the genera Alphacoronaviruses and Betacoronaviruses infect mostly mammals, the Gammacoronavirus infect avian species and members of the Deltacoronavirus genus have been found in both mammalian and avian hosts.

In humans, coronaviruses cause respiratory tract infections that can be mild, such as some cases of the common cold, and others that can be lethal, such as SARS, MERS, and COVID-19.

Coronaviruses are enveloped viruses with a positive-sense single-stranded RNA genome and a nucleocapsid of helical symmetry. The genome size of coronaviruses ranges from approximately 27 to 34 kilobases, the largest among known RNA viruses.

Various species of human coronaviruses are known, such as, without limitation, Human coronavirus OC43 (HCoV-OC43), of the genus β-CoV, Human coronavirus HKU1 (HCoV-HKU1), of the genus β-CoV, Human coronavirus 229E (HCoV-229E), α-CoV, Human coronavirus NL63 (HCoV-NL63), α-CoV, Middle East respiratory syndrome-related coronavirus (MERS-COV), Severe acute respiratory syndrome coronavirus (SARS-COV), Severe acute respiratory syndrome coronavirus 2 (SARS-COV-2).

Coronaviruses vary significantly in risk factor. Some can kill more than 30% of those infected (such as MERS-COV), and some are relatively harmless, such as the common cold. Coronaviruses cause colds with major symptoms, such as fever, and a sore throat, e.g. from swollen adenoids, occurring primarily in the winter and early spring seasons. Coronaviruses can cause pneumonia (either direct viral pneumonia or secondary bacterial pneumonia) and bronchitis (either direct viral bronchitis or secondary bacterial bronchitis). Coronaviruses can also cause SARS.

### Anti-Tumor Effect

The treatment of a tumor disease typically refers to an “anti-tumor effect”. As used herein, an “anti-tumor effect” shall mean inhibiting or slowing down abnormal cell growth and/or migration, or any other medical effect relevant in reducing or preventing cancerous growth. In some embodiments, an “anti-tumor effect” refers to decreasing or not increasing or slowing down the increasing of tumor size.

Citraconic acid directly inhibits the enzyme activity of cis-aconitate-Decarboxylase (ACOD1) which is involved in the last step of synthesis of itaconic acid. In certain tumors, for example, peritoneal tumor preferably, melanoma and ovarian carcinoma, the metabolism of tissue-resident macrophage is altered leading to production of itaconic acid which promotes the tumor growth. In these embodiments, citraconic acid inhibits the enzyme activity of ACOD1 and supresses the tumor growth (reference can be made here to the activity of Itaconic acid, e.g. in Weiss et al., 2018, J Clin Invest. 2018 Aug. 31; 128 (9): 3794-3805.). In addition, citraconic acid inhibits the XPO1 pathway, and it has been shown that XPO1 inhibitors are clinically useful anti-neoplastic medications (e.g., Selinexor for treatment of multiple myeloma).

Tumor diseases include but are not limited to Bladder Cancer, Breast Cancer, Colon and Rectal Cancer, Endometrial Cancer, Kidney Cancer, Leukaemia, Liver Cancer, Lung Cancer, Melanoma, Non-Hodgkin Lymphoma, Pancreatic Cancer, Prostate Cancer, Thyroid Cancer. In some embodiments, the tumor disease may not be associated with inflammation, and is also encompassed by the claimed use of citraconic acid or derivative thereof as described herein.

### Treatment and Immunomodulation

In the present invention “treatment” or “therapy” generally means to obtain a desired pharmacological effect and/or physiological effect. The effect may be prophylactic in view of completely or partially preventing a disease and/or a symptom, for example by reducing the risk of a subject having a disease or symptom or may be therapeutic in view of partially or completely curing a disease and/or adverse effect of the disease.

In the present invention, “therapy” includes treatments of diseases or conditions in mammals, in particular, humans, for example, the following treatments (a) to (c): (a) Prevention of onset of a disease, condition or symptom in a patient; (b) Inhibition of a symptom of a condition, that is, prevention of progression of the symptom; (c) Amelioration of a symptom of a condition, that is, induction of regression of the disease or symptom.

As used herein, “prevent,” and similar words such as “prevented,” “preventing” or “prophylactic” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In one embodiment, “treatment” refers to immunotherapy wherein citraconic acid or derivative is used as an immunomodulator which affects the functioning of the immune system or keeps the homeostasis of immune system. Immunomodulators can adjust the immune response to the correct level, strengthen weak immune systems and control overactive immune systems.

In the pathological inflammatory process, mast cells, monocytes, macrophages, lymphocytes, and other immune cells are first activated. Then the cells are recruited to the site of injury, resulting in the generation of reactive oxygen species (ROS) that damage macromolecules including DNA. At the same time, these inflammatory cells also produce large amounts of inflammatory mediators such as cytokines, chemokines, and prostaglandins. These mediators further recruit macrophages to localized sites of inflammation and directly activate multiple signal transduction cascades and transcription factors associated with inflammation. The NF-κB (nuclear factor kappa B), MAPK (mitogen-activated protein kinase), and JAK (janus kinase)-STAT (signal transducers and activators of transcription) signaling pathways are involved in the development of the classical pathway of inflammation. The transcription factor Nrf2 (NF-E2 p45-related factor 2) regulates the expression of phase II detoxifying enzymes including NADPH, NAD(P)H quinone oxidoreductase 1, glutathione peroxidase, ferritin, heme-oxygenase-1 (HO-1), and antioxidant genes that protect cells from various injuries via their anti-inflammatory effects, thus influencing the course of disease.

In another embodiment, citraconic acid directly inhibits the enzyme activity of cis-aconitate-Decarboxylase (ACOD1) which is involved in the last step of synthesis of itaconic acid. In certain tumors, for example, peritoneal tumors or melanoma and ovarian carcinoma, the metabolism of tissue-resident macrophage is altered leading to production of itaconic acid which promotes the tumor growth. In these embodiments, citraconic acid inhibits the enzyme activity of ACOD1 and supresses the tumor growth.

### Pharmaceutical Compositions

The present invention also relates to a pharmaceutical composition comprising a compound described herein. The invention also relates to pharmaceutically acceptable salts of the compound described herein, in addition to enantiomers and/or tautomers of the compounds described. The term “pharmaceutical composition” refers to a combination of the agent as described herein with a pharmaceutically acceptable carrier. The phrase “pharmaceutically-acceptable” refers to molecular entities and compositions that do not produce a severe allergic or similar untoward reaction when administered to a human. As used herein, “carrier” or “carrier substance” or “diluent” or “excipient” includes without limitation any and all solvents, adjuvant, dispersion media, glidant, sweetening agent, vehicles, coatings, diluents, antibacterial and antifungal agents, flavor enhancer, surfactant, solvent, emulsifier, wetting agent, dispersing agent, isotonic and absorption delaying agents, buffers, carrier solutions, suspensions, colloids, and the like, and which has been also approved by the United States Food and Drug Administration as being acceptable for use in humans or domestic animals. The use of such media and agents for pharmaceutical active substances is well known in the art. Supplementary active ingredients can also be incorporated into the compositions.

A pharmaceutical composition as used herein can be citraconic acid or derivative thereof alone or a composition comprising one or more citraconic acid derivatives, or in combination with one or more pharmaceutical excipients. The pharmaceutical composition as used herein is administered to a subject in an amount which is effective for treating the specific disease or disorder. As used herein “pharmaceutical excipient” shall refer to any given acceptable substance useful in formulating a composition for the drug substance. Examples include, but are not limited to, calcium phosphates, calcium carbonate, calcium sulfate, halites, metallic oxides, sugars, actual sugars, sugar alcohols, artificial sweeteners, modified starch, dried starch, converted starch, cellulose ethers, cellulose esters, CMC, croscarmellose sodium, microcrystalline cellulose, polyethylene glycol, propylene glycol, povidones, petrolatum, mineral waxes, mineral oils, acrylic polymers, fatty alcohols, mineral stearates, glycerin, lipids, other oleochemical excipients and proteins.

The pharmaceutical composition containing the active ingredient may be in a form suitable for oral use, for example, as tablets, chewing tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions. Tablets contain the active ingredient in admixture with non-toxic pharmaceutically acceptable excipients which are suitable for the manufacture of tablets. The tablets may be uncoated or they may be coated by known techniques to delay disintegration and absorption in the gastrointestinal tract and thereby provide a sustained action over a longer period.

The administration of the compositions contemplated herein may be carried out in any convenient manner, including by injection, ingestion, transfusion, topical application or aerosol inhalation. In a preferred embodiment, the compound is administered by ingestion. In some embodiments, “systemic administration” is used, and refers to the administration of a composition that results in broad biodistribution drug or the derivatives thereof within an organism. Systemic administration means exposing a therapeutic amount of an agent to preferred parts of the body. Systemic administration of the composition may be accomplished by any means known in the art, e.g., intravenously, subcutaneously, intraperitoneal. In another embodiment, the compound is administered parenterally. Modes of administration are also considered other than enteral and topical administration, for example by injection, including, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion.

### FIGURES

The following figures are presented to describe particular embodiments of the invention, without being limiting in scope.

### BRIEF DESCRIPTION OF FIGURES

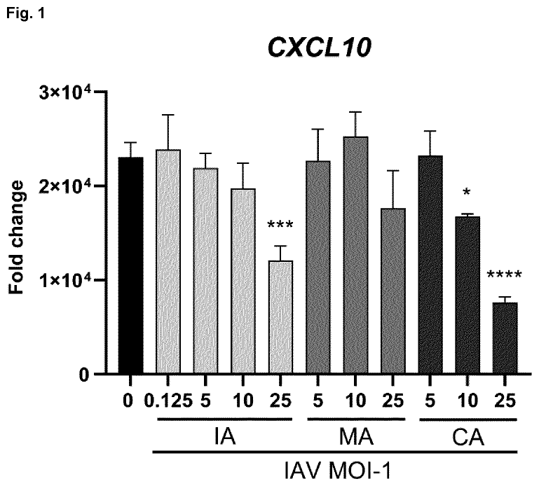

FIG. 1: Anti-inflammatory effect of citraconic acid.

FIG. 2: Anti-oxidative effect of citraconic acid.

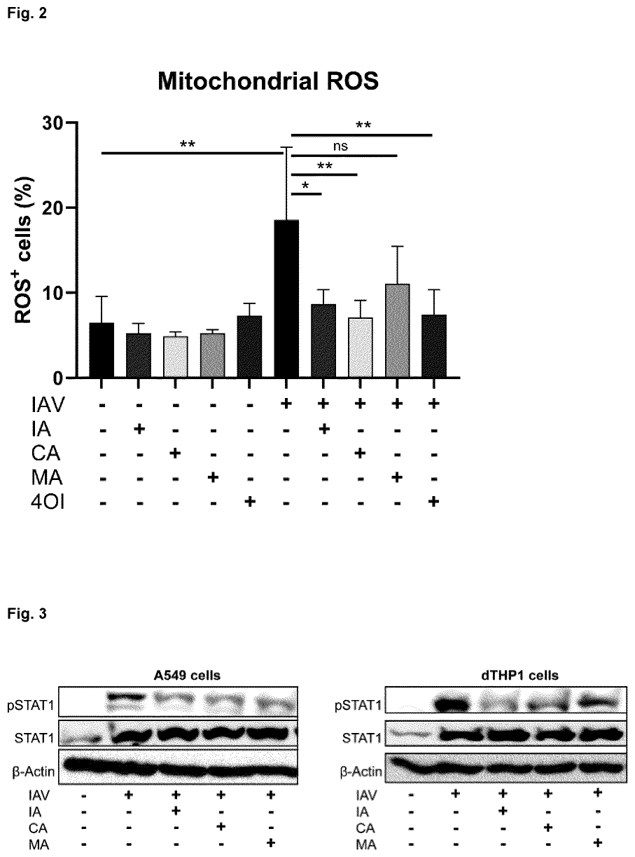

FIG. 3: Reduction of STAT1 phosphorylation by citraconic acid.

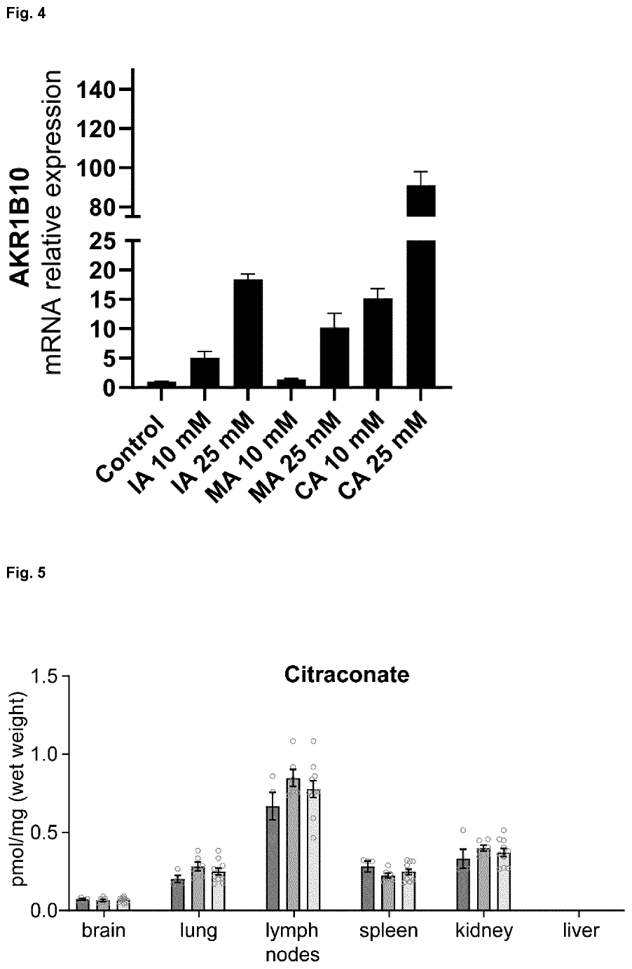

FIG. 4: Induction of Nrf2 signaling by citraconic acid.

FIG. 5: CA concentrations in mouse organs.

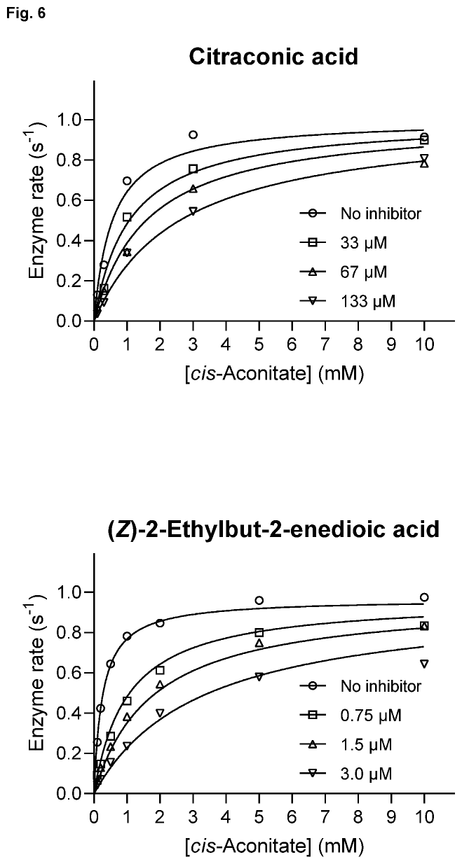

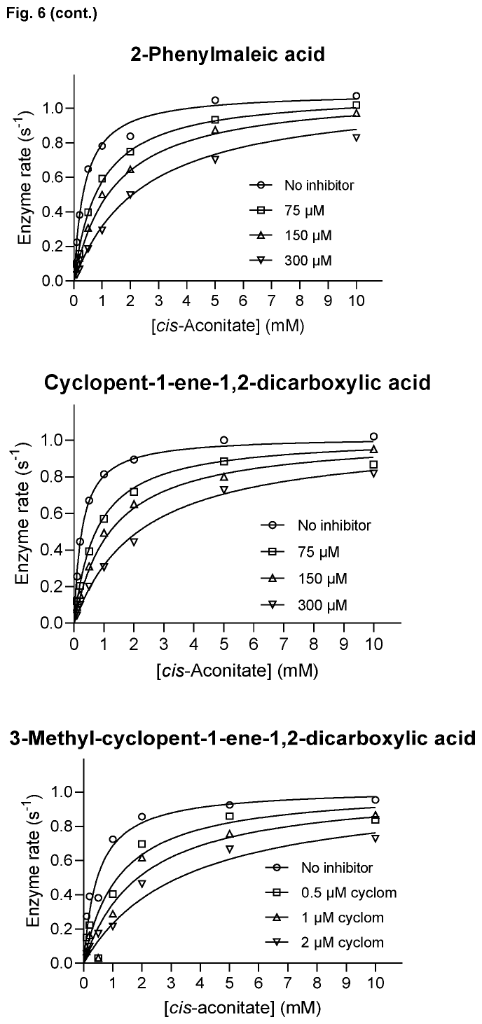

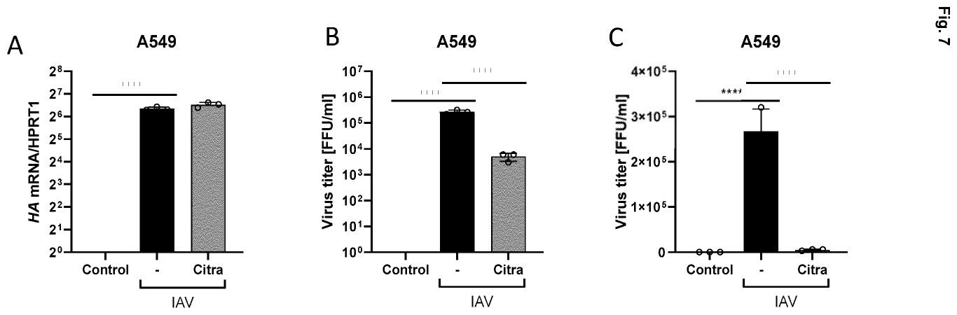

FIG. 6: Five compounds as competitive inhibitors of hACOD1.

FIG. 7: CA inhibits production of influenza A progeny virions, but not viral RNA synthesis.

FIG. 8: Potential covalent binding modes of (A) CA, and (B) 4-octyl citraconate (both in cyan) to CRM1.

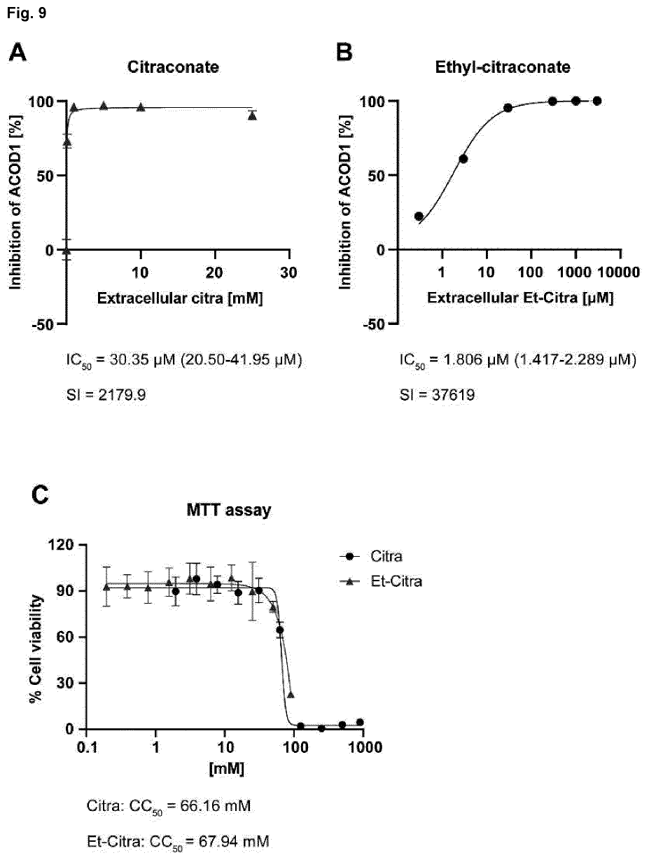

FIG. 9: Ethyl homologue of the methyl group of citraconate enhances ACOD1 inhibition in a cellular assay.

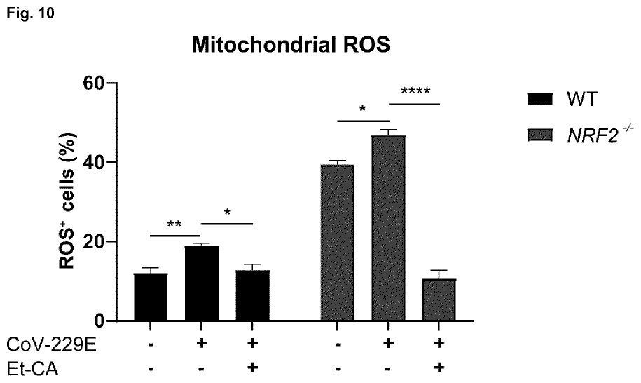

FIG. 10: (Z)-2-Ethylbut-2-enedioic acid maintains anti-oxidative properties.

### DETAILED DESCRIPTION OF THE FIGURES

FIG. 1: Anti-inflammatory effect of citraconic acid. Increasing concentrations of CA and the isomers IA and MA were added to differentiated human macrophage-like cells (dTHP1) that were infected with influenza A virus. Reduction of CXCL10 mRNA (encoding the pro-inflammatory chemokine IP-10) was greatest by CA. Fold change of CXCL10 mRNA expression is shown with reference to uninfected, untreated cells.

FIG. 2: Anti-oxidative effect of citraconic acid. dTHP1 cells were infected with influenza A virus, incubated with 10 mM CA, IA, or MA, and reactive oxygen species (ROS) were measured by flow cytometry. There was significant reduction of ROS by CA and IA.

FIG. 3: Reduction of STAT1 phosphorylation by citraconic acid. A549 and dTHP1 cells were infected with influenza A virus and treated with CA, IA or MA. All three isomers reduced STAT1 phosphorylation (assessed by anti-P-STA1 immunoblot).

FIG. 4: Induction of Nrf2 signaling by citraconic acid. The human keratinocyte cell line HaCaT was incubated with CA, IA or MA and expression of mRNA encoding AKR1B10 (a downstream factor specifically induced by Nrf2 in this cell type) was assessed by RT-qPCR. Induction was greatest by CA, intermediate by IA and lowest by MA.

FIG. 5: CA concentrations in extracted mouse organs of 44 to 46 weeks old C57BL/6J mice. The highest concentrations were detected in lymph nodes, followed by kidney, spleen and lung. Of the three itaconate isomers, CA was the only one that could be quantified in brain, albeit at very low levels. Measurements were made with liquid chromatography—tandem mass spectrometry. n=4 male and n=6 female mice; mean±SEM; values<LLOQ were considered missing values.

FIG. 6: Five compounds as competitive inhibitors of hACOD1. The activity of purified hACOD1 was measured in the presence of increasing concentrations of substrate and the five inhibitors. The data were globally fitted to the Michaelis-Menten equation for competitive inhibition. Resulting values for the inhibition constants (Ki) were 38±10 μM for citraconic acid (Formula III), 60±30 μM for 2-phenylmaleic acid (Formula IX), 235±21 nM for (Z)-2-ethylbut-2-enedioic acid (Formula VIII) and 39±2 μM for cyclopent-1-ene-1,2-dicarboxylic acid (Formula X) and 335 nM for 3-methyl-cyclopent-1-ene-1,2-dicarboxylic acid (Formula XIII).

FIG. 7: Citraconate inhibits production of influenza A progeny virions, but not viral RNA synthesis. The human cell line A549 was infected with influenza A virus (H1N1/34/PR/8M; MOI=1) and viral RNA expression in cells (RT-qPCR) and viral titers in culture supernatants (foci forming assay) were measured 24 hours post infection. A. Viral RNA (hemagglutinin [HA] mRNA; RT-qPCR). B. Viral titers, log 10 scale. C. Viral titers, linear scale.

FIG. 8: Potential covalent binding modes of (A) CA, and (B) 4-octyl citraconate (both in cyan) targeting Cys528 in the nucleoprotein-binding groove of human CRM1 (PDB ID: 6TVO). Lipophilicity protein surface at the nucleoprotein-binding cleft: lipophilic (green), hydrophilic (violet), neutral (white), α-helices (gold).

FIG. 9: Ethyl homologue of the methyl group of citraconate enhances ACOD1 inhibition in a cellular assay. Citraconate and THP1 were differentiated with PMA and then stimulated with LPS/IFNγ for 24 hours in the absence or presence of the indicated concentrations of citraconic acid or its methylated derivative (Z)-2-Ethylbut-2-enedioic acid. ACOD1 activity was assessed by measuring intracellular itaconate concentrations by HPLC-MS/MS as described in (Winterhoff,

Chen et al Metabolites 2021). IC50=inhibitory concentration 50%; CC50 =cytotoxic concentration 50%; SI=selectivity index=CC50/IC50.

FIG. 10: (Z)-2-Ethylbut-2-enedioic acid maintains anti-oxidative properties. Wild-type or NRF2−/−human vascular endothelial cells were infected with human coronavirus 229E (a low pathogenic, seasonal strain), and mitochondrial ROS levels were measured by flow cytometry 24 h after infection. ROS levels are markedly elevated in infected NRF2−/−cells but are reduced to levels seen in uninfected WT cells by co-incubation of the cells with (Z)-2-Ethylbut-2-enedioic acid.

### EXAMPLES

### Example 1: Anti-Inflammatory Effects of Itaconate Isomers

Human monocytic leukaemia cell line THP1 was grown in RPMI 1640 medium supplemented with 10% fetal calf serum and 2 mM L-glutamine and was then differentiated with PMA into a macrophage phenotype (dTHP1). 2×105 cells were infected with influenza A virus (H1N1) strain PR8M at a multiplicity of infection of 1. In the case of treatments, the cells were pre-incubated with the compounds at the indicated concentrations overnight, then incubated with the virus for 2 h in fresh medium to allow virus binding and entry into cells. The infection medium was then replaced with fresh medium containing the treating compound at the indicated concentration. 12 h after infection, cells were washed in buffer, pelleted, and RNA was extracted for subsequent analysis of expression of CXCL10 mRNA (encoding the pro-inflammatory, interferon-induced chemokine IP-10) by real-time quantitative polymerase chain reaction (PCR), using the primer sequences

As can be observed from FIG. 1, increasing concentrations of CA and the isomers IA and MA were added to differentiated human macrophage-like cells (dTHP1) that were infected with influenza A virus. Reduction of CXCL10 mRNA (encoding the pro-inflammatory chemokine IP-10) was greatest by CA, indicating an anti-inflammatory effect of CA.

### Example 2: Measurements of Reactive Oxygen Species (ROS)

dTHP1 Cells were seeded at a density of 2×10⁵ cells per well in a 12-well plate and infected with influenza A virus at an MOI of 1. Mitochondrial ROS were measured 12 h after infection. Cells were incubated for 5 min with medium containing 5 μM of MitoSox Red (mitochondrial superoxide indicator, Invitrogen) and were washed with PBS. Cells were then resuspended in cold PBS and mitochondrial ROS was measured via the phycoerythrin (PE) channel using a BD™ LSR-II flow cytometer.

As can be observed in FIG. 2, dTHP1 cells were infected with influenza A virus, incubated with 10 mM CA, IA, or MA, and reactive oxygen species (ROS) were measured by flow cytometry. The results indicate that there was significant reduction of ROS by CA and IA, compared to controls.

### Example 3: Western Blots for Phosphorylated Stat Protein

2×10⁵ dTHP1 cells were infected with influenza A virus (H1N1) strain PR8M at a multiplicity of infection of 1. 12 h after infection, cells were washed in buffer, pelleted, and protein extracts resolved by gel electrophoresis and transferred to a nylon membrane. Bands corresponding to phosphorylated Stat1 protein were visualized by enhanced chemiluminescense using specific anti-P-STAT1 primary antibody and cromphore-conjugated as secondary antibody.

As can be observed from FIG. 3, A549 and dTHP1 cells were infected with influenza A virus and treated with CA, IA or MA. All three isomers reduced STAT1 phosphorylation when assessed by anti-P-STA1 immunoblot. STAT1 phosphorylation is known to be associated with inflammation, and thus CA represents an effective substance in reducing inflammation.

### Example 4: Induction of Nrf2 Signaling

The immortalized human keratinocyte cell line HaCaT was grown in RPMI 1640 medium (GIBCO Life Technologies, Karlsruhe, Germany) supplemented with 30% FCS, 100 μg/mL pen/strep, 500 μg/mL gentamycin and 1% each Glutamax and nonessential amino acid solution (NEAA; GIBCO Life Technologies, Karlsruhe, Germany). Cells were treated with the indicated concentrations of itaconate, mesaconate and citraconate for 12 hours. Cells were then washed in buffer, pelleted, and RNA was extracted for subsequent analysis of expression of AKR1B10, which is specifically activated by Nrf2 signaling in this cell line. AKR1B10 mRNA expression was determined by RT-qPCR.

As can be observed from FIG. 4, the human keratinocyte cell line HaCaT was incubated with CA, IA or MA and expression of mRNA encoding AKR1B10 (a downstream factor specifically induced by Nrf2 in this cell type) was assessed by RT-qPCR. Induction was greatest by CA compared to both IA and MA.

Based on the data presented herein, CA exhibits surprising and unexpected effects with respect to inhibiting aconitate decarboxylase 1 (ACOD1) activity, inhibiting the expression and/or activity of an immune-response stimulating cytokine, such as CXCL10, exhibiting an anti-oxidative effect (reducing reactive oxygen species (ROS)), and inhibiting phosphorylation of signal transducer and activator of transcription 1 (STAT1). As shown in the examples, despite the similarity in structure between CA, IA and MA, CA appears to show enhanced effects in comparison to these control substances of similar structure. A skilled person would not have expected CA or esters thereof to show this effect, let alone at a greater efficacy in comparison to structurally related compounds.

### Example 5: CA Concentrations in Mouse Organs

Organs were obtained from 44 to 46 weeks old C57BL/6J mice and snap frozen. Frozen organs were weighed in 2 mL FastPrep tubes filled with lysing matrix A (6910, MP Biomedicals, Santa Ana, CA, United States). Subsequently, sample weight was adjusted to 300 mg by addition of 1×PBS and ice cold extraction reagent was added (1.2 mL acetonitrile/methanol 1:1 and 0.1 ml acentonitril/methanol/water 2:2:1 containing internal standards). The organ samples were homogenized at 4° C. using a FastPrep®-24 Instrument (MP Biomedicals, Santa Ana, CA, United States) at a speed of 6.0 for 2×30 s. In between runs, the samples were cooled down again for 5 min. Subsequently, the samples were frozen at −20° C. for 24 h to enhance protein precipitation.

After thawing, samples were centrifuged (10 min, 4° C., 20.000 g) and 1.4 mL of the supernatants was carefully transferred to 2 mL reaction tubes without disturbing the pellet and aspirating the lipid layer. 50 μL calibrator aliquots in surrogate matrix were prepared in 2 mL reaction tubes by adding 250 μL 1×PBS and extraction reagent (1.2 mL acetonitrile/methanol 1:1 and 0.1 mL acetonitrile/methanol/water 2:2:1 containing internal standards) and transferring 1.4 mL of the supernatants after centrifugation. Citraconate concentrations were then measured by LC-MS/MS, using a Nexera chromatography system consisting of a controller (CBM-20A), an autosampler (SIL-30AC), two pumps (LC-30AD), a degasser (DGU 20A5), and a column oven (CTO 20AC, Shimadzu, Japan), coupled to a QTRAP5500 triple quadrupole/linear ion trap mass spectrometer (Sciex, Framingham, MA, USA). Data acquisition and further quantification were performed using the Analyst software 1.7 (Sciex, Framingham, MA, USA).

Based on the data presented herein, surprisingly, the highest concentrations were detected in lymph nodes, followed by kidney, spleen and lung. Of the three itaconate isomers, CA was the only one that could be quantified in brain, albeit at very low levels.

### Example 6: Synthesis and Analytical Chemistry of Citraconic Acid Esters

In order to assess whether citraconic acid esters also exhibit the desired and therapeutically relevant effects, a series of citraconic acid esters was generated for further testing.

Starting materials and solvents were purchased from commercial suppliers and were used without further purification. Reaction progress was monitored using TLC silica gel 60 F₂₅₄ aluminum sheets, and visualization was accomplished by UV at 254 nm. Flash chromatography was performed using silica gel 60 Å (40-63 μm). Melting points were determined on a Stuart Scientific melting point apparatus SMP30 (Bibby Scientific, UK). NMR spectra were recorded on a Bruker Avance Neo 500 MHz with CryoProbe Prodigy system (¹H, 500 MHz; ¹³C, 126 MHZ) at 298 K. Chemical shifts were recorded as δ values in ppm units by reference to the hydrogenated residues of deuterated solvent as internal standard (DMSO-d₆, δ=2.50, 39.51). Purity of all compounds used in biological assays was ≥95% as determined on a Dionex UltiMate 3000 UHPLC⁺focused/Thermo Scientific Q Exactive Focus Orbitrap LC-MS/MS system (Thermo Fisher Scientific, Dreieich, Germany).

Given the decent permeability of the free dicarboxlytes, we synthesized less polar citraconate derivatives to potentially improve cell permeability, namely the dimethyl ester 1 and the regioisomeric mono octyl esters 2 and 3 (Scheme 1, below).

Compound 1 was prepared via a mild and rapid methylation of the citraconate using TMS diazomethane. The mono octyl esters 2 and 3 were obtained by heating citraconic anhydride and n-octanol at 110°°C. for 4 h in a neat reaction. The citraconate monoesters were separated by flash chromatography. Notably, both 1-octyl (2) and 4-octyl (3) isomers were formed in a nearly equal ratio, indicating no regioselectivity under the reaction conditions.

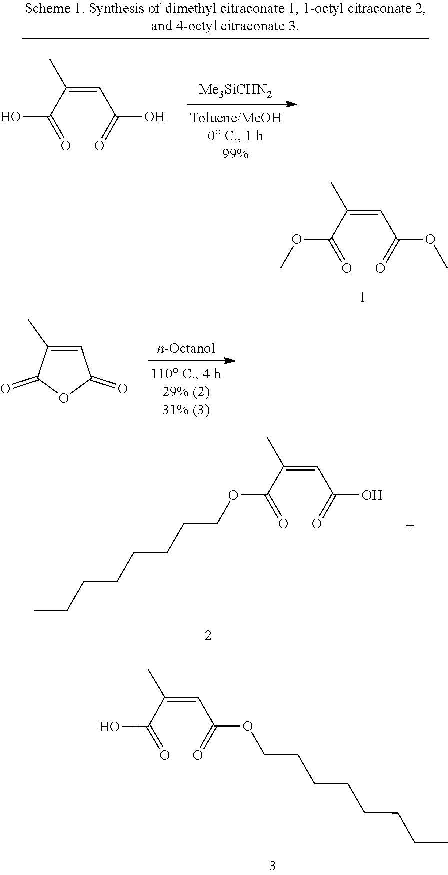

### Dimethyl citraconate (1, WAM474)

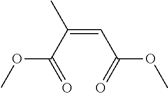

To a stirred ice-cooled solution of citraconic acid (130 mg, 1 mmol) in a mixture of toluene/methanol (9:4, 13 mL), (trimethylsilyl) diazomethane (2 M solution in diethyl ether) (2 mL, 4 mmol) was added cautiously. The reaction mixture was stirred at 0° C. for 1 h. The solvent and volatiles were removed under reduced pressure in a water bath at 30° C. The obtained material was sufficiently pure as indicated by UPLC-HRMS chromatograms and NMR spectra.

Yield 99%; yellow liquid; ¹H NMR (500 MHZ, DMSO-d₆) δ 6.08 (s, 1H), 3.70 (s, 3H), 3.64 (s, 3H), 2.00 (s, 3H); ¹³C NMR (126 MHz, DMSO-d₆) δ 168.67, 164.92, 145.30, 120.70, 52.10, 51.64, 19.75; HRMS (ESI+) calcd. for C₇H₁₁O₄ [M+H]⁺: 159.0657, found: 159.0651; t_{R}=5.10 min.

### 1-Octyl citraconate (2, WAM476)

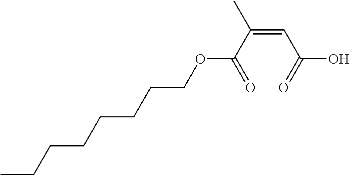 [SMILES: CCCCCCCCOC(=O)/C(C)=C\C(=O)O]

To citraconic anhydride (1.12 g, 10 mmol) in a 10 mL Pyrex pressure tube, 1-octanol (1.37 g, 10.5 mmol) was added. The reaction mixture was stirred at 110° C. for 4 h, then it was allowed to cool to rt. The mixture was poured into water (50 mL) and was extracted by EtOAc (2×50 mL). Organic layers were dried (MgSO₄), filtered, and solvent was removed under reduced pressure. The residue comprised a mixture of 1- and 4-octyl regioisomers that were separated by flash chromatography (SiO₂, petroleum ether 40/60-EtOAc=5:1 as a mobile phase in an isocratic elution). The 1-octyl ester (2) eluted first followed by the 4-octyl isomer (3).

Yield 29%; white solid; mp 37-38° C.; ¹H NMR (500 MHZ, DMSO-d₆)δ 12.59 (br s, 1H), 5.93 (s, 1H), 4.08 (t, J=6.6 Hz, 2H), 1.95 (s, 3H), 1.58 (p, J=7.0 Hz, 2H), 1.24 (m, 10H), 0.85 (t, J=7.1 Hz, 3H); ¹³C NMR (126 MHZ, DMSO-d₆) δ 168.62, 165.89, 144.35, 121.84, 64.69, 31.30, 28.68, 28.67, 27.85, 25.39, 22.16, 19.88, 14.03; HRMS (ESI⁺) calcd. for C₁₃H₂₃O₄[M+H]⁺: 243.1596, found: 243.1587; t_{R}=6.84 min.

### 4-Octyl citraconate (3, WAM475)

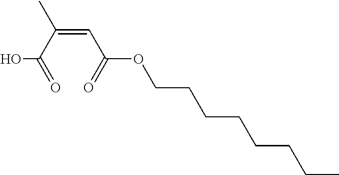 [SMILES: CCCCCCCCOC(=O)/C=C(/C)C(=O)O]

Compound 3 was obtained from the same reaction for compound 2. It was separated by flash chromatography (SiO₂, petroleum ether 40/60-EtOAc=5:1) in the more polar fractions.

Yield 31%; colorless liquid; ¹H NMR (500 MHZ, DMSO-d₆)δ 13.03 (br s, 1H), 5.93 (s, 1H), 4.02 (t, J=6.6 Hz, 2H), 1.97 (s, 3H), 1.55 (p, J=7.0 Hz, 2H), 1.24 (m, 10H), 0.85 (t, J=7.0 Hz, 3H); ¹³C NMR (126 MHZ, DMSO-d₆) δ 169.81, 164.68, 146.37, 119.34, 64.12, 31.28, 28.69, 28.67, 28.04, 25.41, 22.16, 20.13, 14.03; HRMS (ESI⁺) calcd. for C₁₃H₂₃O₄ [M+H]⁺: 243.1596, found: 243.1588; t_{R}=6.85 min.

Preliminary results indicate the esters 1, 2 and 3 to exhibit comparable biological properties as citraconic acid.

### Example 7: Assessment of hACOD1 Enzyme Activity by In Vitro Assay and Inhibition by CA, (Z)-2-ethylbut-2-enedioic Acid 2-phenylmaleic Acid, cyclopent-1-ene-1,2-dicarboxylic Acid and 3-methyl-cyclopent-1-ene-1,2-dicarboxylic Acid

Increased ACOD1 inhibition was shown for a CA derivative 3-methyl-cyclopent-1-ene-1,2-dicarboxylic acid (Formula XIII) in a cell-free assay, showing comparable inhibition to (Z)-2-ethylbut-2-enedioic acid (Formula VIII). Below is the updated list of structural formulas of CA and now 4 derivatives, as well as the ACOD1 inhibition assay with the 5 compounds.

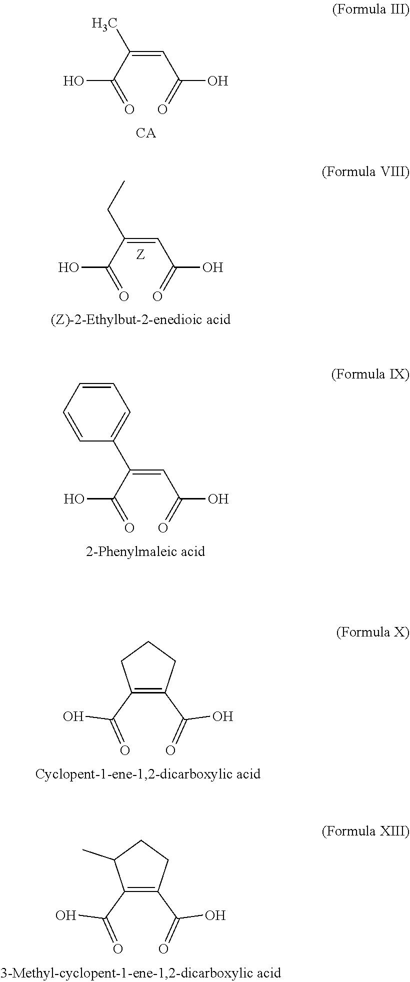

The activity of purified hACOD1 was measured in the presence of increasing concentrations of substrate and the five inhibitors. The data was globally fitted to the Michaelis-Menten equation for competitive inhibition.

Resulting values for the inhibition constants (Ki) were 38±10 μM for citraconic acid, 60+30 μM for 2-phenylmaleic acid, 235±21 nM for (Z)-2-ethylbut-2-enedioic acid and 39±2 μM for cyclopent-1-ene-1,2-dicarboxylic acid and 335 nM for 3-methyl-cyclopent-1-ene-1,2-dicarboxylic acid.

The enzyme was prepared as described (Chen, F. et al., 2019, Proc. Natl. Acad. Sci. U. S. A. 116:20644-20654). cis-Aconitic acid (Sigma-Aldrich #A3412), citraconic acid (Acros Organics #110430050), (Z)-2-ethylbut-2-enedioic acid (Aurora Fine Chemicals #132.916.113), 2-phenylmaleic acid (Activate Scientific #AS113764), cyclopent-1-ene-1,2-dicarboxylic acid (Key Organics ID 6W-0249) and 3-methyl-cyclopent-1-ene-1,2-dicarboxylic acid (Sigma-Aldrich #R261629) were obtained from commercial sources. The compounds were dissolved in water, neutralized with NaOH and stored at −20° C. For the assay, 125 μL 0.2 M sodium phosphate buffer, pH 6.5, was mixed on ice with 5 μL enzyme, 10 μl inhibitor (or 10 μl water) and 10 μL substrate (cis-aconitic acid). The following combinations of enzyme amount and substrate concentration were used for CA: 2 μg enzyme and 0.1 mM substrate, 2 μg and 0.3 mM, 3 Ξg and 1 mM, 5 μg and 3.0/10.0 mM. For the other inhibitors, 2 μg enzyme and 0.1/0.2/0.5 mM substrate, 3 μg and 1.0/2.0 mM, 5 μg and 5.0/10.0 mM. Incubation for 10 min at 37° C. was immediately followed by heat inactivation at 95° C. for 3 min. Protein precipitate was pelleted by centrifugation for 1 h. Supernatants were acidified with 100 μL 100 mM H₃PO₄. Itaconic acid was measured by HPLC (Shodex Shodex RSpak DE-413 column, 1 mL/min 10 mM H3PO4, detection at 210 nm). The resulting curves of enzyme rate over substrate concentration were fitted using GraphPad Prism 8 with the Michaelis-Menten equation for competitive inhibition, rate=k_{cat}·[S]/(K_{M}·(1+[I] /Kᵢ)+[S]), with the independent variables inhibitor concentration [I] and substrate concentration [S].

### Example 8: Measure of Activity of hACOD1 in the Presence of Increasing Concentrations of Substrate and the Four Compounds in Example 7

As shown in FIG. 6, the activity of purified hACOD1 was measured in the presence of increasing concentrations of substrate and the four inhibitors. The data were globally fitted to the Michaelis-Menten equation for competitive inhibition. Resulting values for the inhibition constants (Ki) were 38±10 μM for citraconic acid, 60±30 μM for 2-phenylmaleic acid, 235±21 nM for (Z)-2-ethylbut-2-enedioic acid and 39±2 μM for cyclopent-1-ene-1,2-dicarboxylic acid (n=3).

### Example 9: Anti-Viral Effect of CA

As shown in Figure, CA inhibits production of influenza A progeny virions, but not viral RNA synthesis. The human cell line A549 was infected with influenza A virus (H1N1/34/PR/8M; MOI=1) and viral RNA expression in cells (RT-qPCR) and viral titres in culture supernatants (foci forming assay) were measured 24 hours post infection. A. Viral RNA (hemagglutinin [HA] mRNA; RT-qPCR) is not influenced by CA. B. Viral titres are significantly reduced upon treatment of CA, log 10 scale. C. Viral titres are shown in linear scale.

### Example 10: Docking Study of CA and Derivatives Into Human Nuclear Export Factor CRM1

All computational work was performed using Molecular Operating Environment (MOE), version 2020.09, Chemical Computing Group ULC, 910-1010 Sherbrooke St. W. Montreal, Quebec, H3A 2R7, Canada.

Preparation of ligands and protein structures: The 2D structures of citraconic acid, and 4-octyl citraconate were sketched using ChemDraw professional 19.0 and were imported into the MOE window. The compounds were subjected to an energy minimization up to a gradient of 0.001 kcal mol-1 Å2 using the MMFF94x force field and R-field solvation model, then they were saved as mdb file. The predominant protonation status of the compounds in aqueous medium at pH 7 was calculated via the compute | molecule | wash command in the database viewer window. X-ray crystal structures of the human chromosome region maintenance 1 (CRM1) in complex with leptomycin B (PDB ID: 6TVO) were used for the molecular-docking study. Potential was set up to Amber10: EHT as a force field and R-field for solvation. Addition of hydrogen atoms, removal of water molecules farther than 4.5 Å from ligand or receptor, correction of library errors, and tethered energy minimization of binding site were performed via the QuickPrep module.

Structural modelling: Covalent docking was performed in the leptomycin B binding site of CRM1. The Cys528 residue was selected as the reactive site. The 1,4-Michael addition was set as the reaction. Placement trials were set to 100 poses with an induced fit refinement. Final scoring function was GBVI/WSA dG with 10 poses.

Results and discussion: We carried out a covalent docking study using the human nuclear export protein chromosomal region maintenance 1 (CRM1), a potential antiviral target of itaconate derivatives and other Michael acceptors. Results showed that citraconate, the itaconate isomer, and 4-octyl citraconate efficiently undergo Michael 1,4-addition reaction with the Cys528 residue of CRM1 (Table 1 and FIG. 8). Both covalently bound compounds establish hydrogen bonds between their free carboxyl groups and Lys537 and Lys568. Interestingly, 4-octyl citraconate shows more favourable binding through hydrophobic interactions between the octyl chain and IIe521, Leu525, Met545, Phe561, Val565 and Leu569 in the nucleoprotein-binding site.

TABLE 1
Covalent binding energies of citraconate
and 4-octyl citraconate to human CRM1

| Compound | hCRM1 Binding energy score (S) (kcal/mol) |
|---|---|
| Citraconate | −1.4 to −3.0 |
| 4-Octyl citraconate | −4.6 to −5.5 |

### Example 11: Increased ACOD1 Inhibition by (Z)-2-ethylbut-2-enedioic Acid (“ethylcitraconic Acid”) in a Cellular Model

Detection of increased ACOD1 inhibition caused by (Z)-2-ethylbut-2-enedioic acid (“ethylcitraconic acid”; Formula VIII) is shown using a cellular model (also demonstrated in a cell-free in vitro assay above). As can be seen from FIG. 9, the selectivity index increases, as the cytotoxicity remains the same. Methylation of the internal carbon of citraconate thus enhances ACOD1 inhibition in a cellular assay. Citraconate and THP1 were differentiated with PMA and then stimulated with LPS/IFNγ for 24 hours in the absence or presence of the indicated concentrations of citraconic acid or its methylated derivative (Z)-2-Ethylbut-2-enedioic acid. ACOD1 activity was assessed by measuring intracellular itaconate concentrations by HPLC-MS/MS as described in Winterhoff, Chen et al (Metabolites 2021).

### Example 12: Maintainence of Anti-Oxidative Properties of (Z)-2-ethylbut-2-enedioic Acid

Maintenance of anti-ROS activity of (Z)-2-ethylbut-2-enedioic acid (“ethylcitraconic acid”; Formula VIII) is shown in a model of infection of cells by a human coronavirus 229E, a low pathogenic CoV strain. Despite the methylation of CA evident in Formel VIII (a methylation that could theoretically lead to loss of electrophilicity), the compound (Z)-2-Ethylbut-2-enedioic acid maintains anti-oxidative properties. Wild-type or NRF2−/−human vascular endothelial cells were infected with human coronavirus 229E (a low pathogenic, seasonal strain), and mitochondrial ROS levels were measured by flow cytometry 24 h after infection. ROS levels are markedly elevated in infected NRF2−/−cells but are reduced to levels seen in uninfected WT cells by co-incubation of the cells with (Z)-2-Ethylbut-2-enedioic acid.

1. (canceled)

2. A method for treating a medical condition associated with inflammation, comprising administering citraconic acid or derivative thereof, according to formula VII to a subject in need thereof, 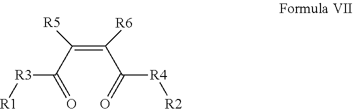
wherein R1 and R2 are, independently, H, a branched or straight chain alkyl, alkenyl, alkynyl, aryl, cycloalkyl or alkoxy group of C1-C12, wherein R1 and/or R2 is optionally substituted with halogen, alkoxy, hydroxy, amine or C1-C8 alkyl,
wherein R3 and R4 are, independently, a heteroatom,
wherein R5 and R6 are, independently, H, a branched or straight chain alkyl, alkenyl, alkynyl, aryl, heteroaryl, cycloalkyl or alkoxy group of C1-C12, wherein R5 and/or R6 is optionally substituted with halogen, alkoxy, hydroxy, amine or C1-C8 alkyl, and
wherein optionally, R5 is fused to R6 to form a cyclic structure.

3. The method according to claim 2, wherein the citraconic acid or derivative thereof is according to formula I, 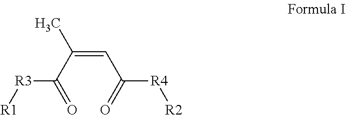
wherein R1 and R2 are, independently, H, a branched or straight chain alkyl, alkenyl, alkynyl, aryl, cycloalkyl or alkoxy group of C1-C12, wherein R1 and/or R2 is optionally substituted with halogen, alkoxy, hydroxy, amine or C1-C8 alkyl, and
wherein R3 and R4 are, independently, a heteroatom.

4. (canceled)

5. The method according to claim 2 wherein the medical condition associated with inflammation comprises chronic inflammation.

6. The method according to claim 2 wherein the medical condition associated with inflammation comprises acute inflammation.

7. The method according to claim 2, wherein the medical condition associated with inflammation comprises immune paralysis.

8. The method according to claim 2, wherein the medical condition comprising acute inflammation is a disease of the lung.

9. The method according to claim 2, wherein the medical condition associated with inflammation is a viral infection.

10. The method according to claim 2, wherein the medical condition associated with inflammation is a severe acute inflammatory syndrome.

11. The method according to claim 2, wherein the medical condition is fever, a tumor disease, a neurodegenerative disease, or pain.

12. The method according to claim 5, wherein the medical condition comprising chronic inflammation is selected from the group consisting of cardiovascular disease, diabetes, autoimmune disease, rheumatic disease, allergy, arthritis, bowel diseases, psoriasis, pulmonary disease, graft vs host disease (GVHD) and transplant rejection.

13. (canceled)

14. The method according to claim 2 wherein said treatment:
a. induces an Nrf2 anti-inflammatory signaling pathway,
b. inhibits synthesis of itaconic acid,
c. inhibits aconitate decarboxylase 1 (ACOD1) activity,
d. inhibits expression and/or activity of an immune-response stimulating cytokine, comprising CXCL10,
e. exhibits an anti-oxidative effect (reducing reactive oxygen species (ROS),
f. inhibits phosphorylation of signal transducer and activator of transcription 1 (STAT1), And/or and/or
g. interacts with chromosomal region maintenance 1 (CRM1), wherein Cys528 in the nucleoprotein-binding groove of CRM1 is targeted.

15. (canceled)

16. (canceled)

17. (canceled)

18. (canceled)

19. A citraconic acid ester, according to formula V or formula VI

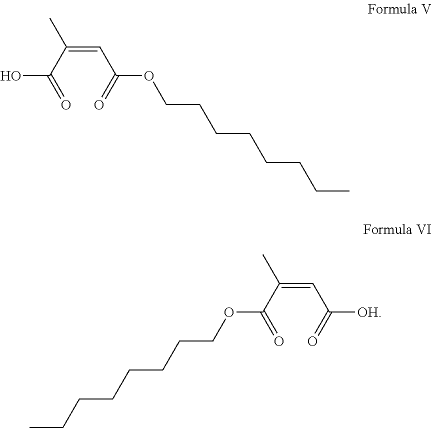

20. (canceled)

21. The method according to claim 2, wherein said citraconic acid or derivative thereof is present in a pharmaceutical composition with one or more pharmaceutical excipients.

22. The method according to claim 2,
wherein R1 and R2 are, independently, C1-C8 alkyl,
wherein R1 and/or R2 is optionally substituted with halogen, alkoxy, hydroxy, amine or C1-C8 alkyl,
wherein R3 and R4 are, independently, O, S or N,
wherein R5 and R6 are, independently, C1-C4 alkyl or phenyl,
wherein R5 and/or R6 is optionally substituted with halogen, alkoxy, hydroxy, amine or C1-C8 alkyl, and
wherein optionally, R5 is fused to R6 to form a cyclic 5- or 6-membered structure.

23. The method according to claim 3,
wherein R1 and R2 are, independently, C1-C8 alkyl,
wherein R1 and/or R2 is optionally substituted with halogen, alkoxy, hydroxy, amine or C1-C8 alkyl, and
wherein R3 and R4 are, independently, O, S or N.

24. The method according to claim 9, wherein the viral infection is an influenza virus, zika virus or coronavirus infection.

25. The method according to claim 9, wherein the treatment inhibits replication of the viral genome.

26. The method according to claim 10, wherein the severe acute inflammatory syndrome is a severe acute respiratory syndrome (SARS), sepsis, septic shock, multiple organ failure or a cytokine release syndrome.

27. The method according to claim 2, for inhibiting synthesis of itaconic acid and/or inhibiting aconitate decarboxylase 1 (ACOD1) activity in the treatment of a medical condition associated with inflammation.

28. The method according to claim 2, wherein the citraconic acid or derivative thereof is according to: [chemical structure]
